SEQUENCE LISTING (5 sequences)

SEQ ID NO: 1 - protein, 6 aa, Artificial Sequence
  Chemically synthesized
CGRGDS

SEQ ID NO: 2 - protein, 7 aa, Artificial Sequence
  Chemically synthesized
CGYIGSR

SEQ ID NO: 3 - protein, 11 aa, Artificial Sequence
  Chemically synthesized
CGPQGIWGQGC

SEQ ID NO: 4 - protein, 8 aa, Artificial Sequence
  Chemically synthesized
GPQGIAGQ

SEQ ID NO: 5 - protein, 8 aa, Artificial Sequence
  Chemically synthesized
IPVSLRSG

(19) United States
(12) Patent Application Publication
(10) Pub. No.: US 2020/0347359 A1
(43) Pub. Date: Nov. 5, 2020

(54) 3D in vitro Models of Lung Tissue

(71) Applicant: THE REGENTS OF THE UNIVERSITY OF COLORADO, A BODY CORPORATE, Denver, CO (US)

(72) Inventors: Chelsea M. MAGIN, Denver, CO (US); Tyler J. D'OVIDIO, Lakewood, CO (US); Nicole Joanne DARLING, Littleton, CO (US)

(21) Appl. No.: 16/960,445

(22) PCT Filed: Jan. 8, 2019
(86) PCT No.: PCT/US2019/012722

§ 371 Date: Jul. 7, 2020

Related U.S. Application Data
(60) Provisional application No. 62/614,702, filed on Jan. 8, 2018.
(60) Provisional application No. 62/700,786, filed on Jul. 19, 2018.

(51) Int. Cl. C12N 5/071; C08G 65/48; C08F 290/14; C08J 3/075; C12N 5/00

(52) U.S. Cl. CPC C12N 5/0688; C08G 65/48; C08F 290/142; C08J 3/075; C12N 5/0062; C12N 2539/00; C08J 2351/08; C12N 2506/45; C12N 2533/30; C12N 2533/50; C12N 2537/10; C12N 2513/00

(57) ABSTRACT

The invention relates to the discovery of tissue mimicking constructs and compositions that can be used to study the growth and development of cells in vitro. In certain embodiments, the invention provides methods of culturing cells on the tissue mimicking polymer microspheres. In other embodiments, the invention provides methods of treating a disease or disorder using the compositions and constructs of the invention.

### CROSS REFERENCE TO RELATED APPLICATIONS

The present application claims priority under 35 U.S.C. § 119(e) to U.S. Provisional Patent Applications No. 62/614,702, filed Jan. 8, 2018, and No. 62/700,786, filed Jul. 19, 2018, all of which are incorporated herein by reference in their entireties.

### BACKGROUND OF THE INVENTION

Nearly 100,000 patients are impacted by idiopathic pulmonary fibrosis (IPF) in the United States alone with approximately 34,000 new diagnoses every year. IPF is a chronic, progressive and life-threatening lung disease that is most prevalent in elderly populations—average age at diagnosis is 57-75 years. Although most patients with IPF succumb to respiratory failure within 3-5 years, the only clinically available therapeutic treatments do not cure the disease. As the average age of the U.S. population continues to increase, it will be imperative for scientists and clinicians to work together to identify new targets to halt or reverse IPF.

Mechanistic studies of the disease indicate that repeated injury to alveolar epithelial cells leads to a profibrotic phenotype that initiates an aberrant wound-healing response in surrounding fibroblasts through secretion of mediators capable of inducing fibroblast migration, proliferation and activation, including TGF-β, platelet-derived growth factor (PDGF) and connective tissue growth factor (CTGF). Over time, these interactions result in pulmonary fibrosis characterized by local tissue stiffening and excess extracellular matrix (ECM) deposition. Current methods of studying lung cell cultures are limited, in that they rely either on 2D scaffolds or naturally derived materials such as Matrigel or collagen, which are limited in their usefulness, because they cannot be modified or systematically tested and improved.

There remains a need in the art for materials and methods for culturing lung cells in 3D bio-inspired microenvironments that accurately mimic the alveoli of a healthy subject as well as a diseased subject. The present invention addresses these needs.

### BRIEF SUMMARY OF THE INVENTION

In one aspect, the invention provides a method of culturing cells in an in vitro tissue model. In another aspect, the invention provides a polymer microsphere composition comprising at least one multifunctional monomer; at least one peptide segment; and at least one degradable crosslinker. In yet another aspect, the invention provides an aggregated alveoli-like structure comprising the polymer microsphere composition of the invention. In yet another aspect, the invention provides a method of treating a disease or disorder in a subject, the method comprising administering a composition of the invention or a structure of the invention to the subject.

In certain embodiments, the method comprises incubating cells seeded in a uniformly dispersed polymer microsphere composition. In certain embodiments, the method comprises aggregating portions of the uniformly dispersed polymer microsphere composition to form alveoli-like clusters. In certain embodiments, the method comprises encapsulating and incubating the alveoli-like clusters in an encapsulating matrix material.

In certain embodiments, the polymer microspheres comprise at least one multifunctional monomer, at least one peptide segment, and at least one degradable crosslinker.

In certain embodiments, the encapsulating matrix material comprises at least one multifunctional monomer, at least one crosslinker, and at least one peptide segment.

In certain embodiments, at least one crosslinker in the encapsulating matrix material and at least one degradable crosslinker in the polymer microspheres are different.

In certain embodiments, at least one crosslinker in the encapsulating matrix material and at least one degradable crosslinker in the polymer microspheres are the same.

In certain embodiments, the at least one crosslinker in the encapsulating matrix material is same as the at least one degradable crosslinker in the polymer microspheres.

In certain embodiments, the at least one multifunctional monomer is each independently selected from the group consisting of functionalized poly(ethylene glycol), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), acrylate-functionalized gelatin, methacrylate-functionalized gelatin, acrylate-functionalized hyaluronic acid, and methacrylate-functionalized hyaluronic acid.

In certain embodiments, the at least one multifunctional monomer is each independently functionalized with at least one functional moiety selected from the group consisting of acrylate, methacrylate, norbornene, thiol, azide, alkene, alkyne, oxime, hydrozone, isocyanate, tetrazine, maleimide, vinyl sulphone, dibenzocyclooctyne and NHS-ester.

In certain embodiments, the at least one multifunctional monomer is each independently functionalized with at least two, at least three, at least four, or at least eight functional moieties.

In certain embodiments, the at least one multifunctional monomers in the polymer microspheres and the at least one multifunctional monomers in the encapsulating matrix are independently selected and may be either the same or different.

In certain embodiments, the at least one multifunctional monomer is a compound of Formula (IA):

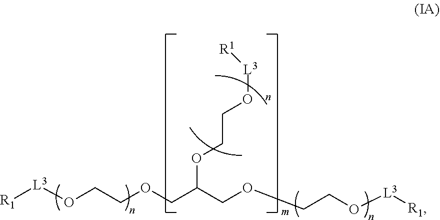

wherein, each instance of L³ independently comprises a linkage selected from the group consisting of a bond,

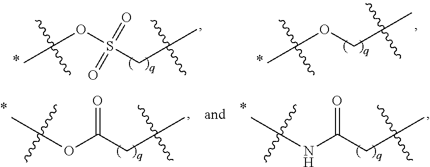

wherein the * side of the linkage is bound to the monomer and the opposite side is bound to R¹, and wherein q is an integer selected from 0 to 6; each instance of R¹ independently comprises a functionality selected from the group consisting of acrylate, methacrylate, norbornene, thiol, azide, alkene, alkyne, oxime, hydrozone, isocyanate, tetrazine, maleimide, vinyl sulphone, dibenzocyclooctyne, NHS-ester. N C

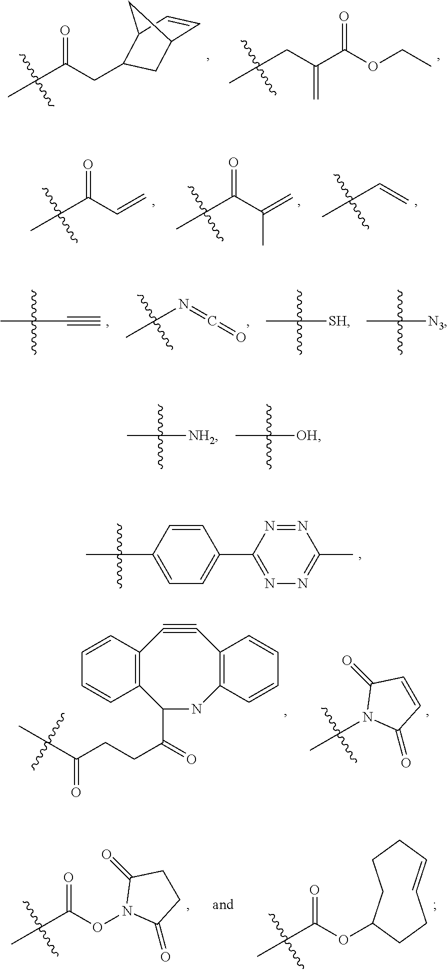

m is an integer from 0 to 10; and n is an integer from 1 to 500.

In certain embodiments, the at least one peptide segment is a segment from at least one protein selected from the group consisting of matrisome protein and matrisome-associated protein. In certain embodiments, the matrisome protein comprises at least one selected from the group consisting of glycoproteins, proteoglycans and collagen. In certain embodiments, the matrisome-associated protein comprises at least one selected from the group consisting of secreted factors, extracellular matrix-affiliated proteins and extracellular matrix regulators.

In certain embodiments, the at least one peptide segment is a segment from at least one protein selected from the group consisting of collagen, elastin, fibronectin, laminin, fibrillin, tenascin, vitronectin, serpin, asporin and osteonectin.

In certain embodiments, the at least one peptide segment is a synthetic peptide segment that mimics a segment of at least one protein selected from the group consisting of collagen, elastin, fibronectin, laminin, fibrillin, tenascin, vitronectin, serpin, asporin, and osteonectin.

In certain embodiments, at least one peptide segment comprises CGRGDS (SEQ ID NO:1).

In certain embodiments, at least one peptide segment comprises CGYIGSR (SEQ ID NO:2).

In certain embodiments, the polymer microspheres and/or the encapsulating matrix material further comprises an additional peptide segment comprising CGRGDS.

In certain embodiments, the polymer microspheres and/or the encapsulating matrix material further comprises an additional peptide segment comprising CGYIGSR.

In certain embodiments, the at least one peptide segment in the polymer microspheres and the at least one peptide segment in the encapsulating matrix are independently selected and may be either the same or different.

In certain embodiments, the cells are selected from the group consisting of basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells, airway or bronchial epithelial cells and cell lines comprising A549, MLE-12 and/or 3T3 fibroblasts.

In certain embodiments, the at least one degradable crosslinker is an enzyme-degradable crosslinker, a protease-degradable crosslinker, a photodegradable crosslinker, and/or a biodegradable crosslinker. In certain embodiments, at least one degradable crosslinker is a matrix metalloprotease (MMP) degradable crosslinker.

In certain embodiments, wherein the at least one degradable crosslinker is degraded through exposure to at least one selected from visible light (380 nm-760 nm) photoexcitation and ultraviolet (UV) light photoexcitation (100 nm-380 nm).

In certain embodiments, the at least one degradable crosslinker comprises at least one selected from the group consisting of ortho-nitrobenzyl moieties, coumarin, azobenzene, rotaxane, aromatic disulfides, poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), spermine, 2,2′-(ethylenedioxy)bis (ethylamine) (EDBE), CGPQGIWGQGC (SEQ ID NO:3), GPQGIAGQ (PCL-1; SEQ ID NO:4) and IPVSLRSG (PCL-2; SEQ ID NO:5).

In certain embodiments, the at least one degradable crosslinker is at least one peptide selected from the group consisting of CGPQGIWGQGC, GPQGIAGQ (PCL-1), and IPVSLRSG (PCL-2).

In certain embodiments, the at least one degradable crosslinker is a compound of Formula (II):

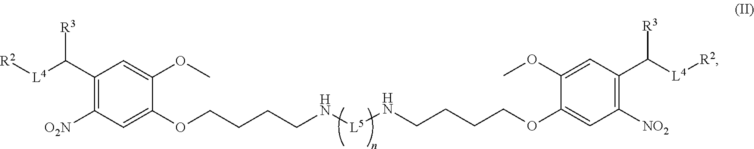

wherein: each instance of L⁴ independently comprises a linkage having a structure selected from the group consisting of:

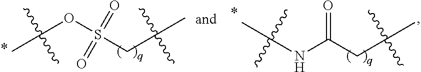

wherein the * side of the linkage is bound to the monomer and the opposite side is bound to R², and wherein q is an integer selected from 0 to 6; L⁵ is a polymeric linker moiety comprising at least one selected from the group consisting of polyethylene glycol (PEG), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), and poly-D,L-lactic acid-p-dioxanone-polyethylene glycol block copolymer (PLA-DX-PEG); each instance of R² independently comprises a functionality selected from the group consisting of acrylate, methacrylate, norbornene, thiol, tetrazine, amine, dibenzocyclooctyne, maleimide, succinimide, trans-cyclooctene, azide, alkene, alkyne, oxime, hydrazone, alcohol, isocyanate,

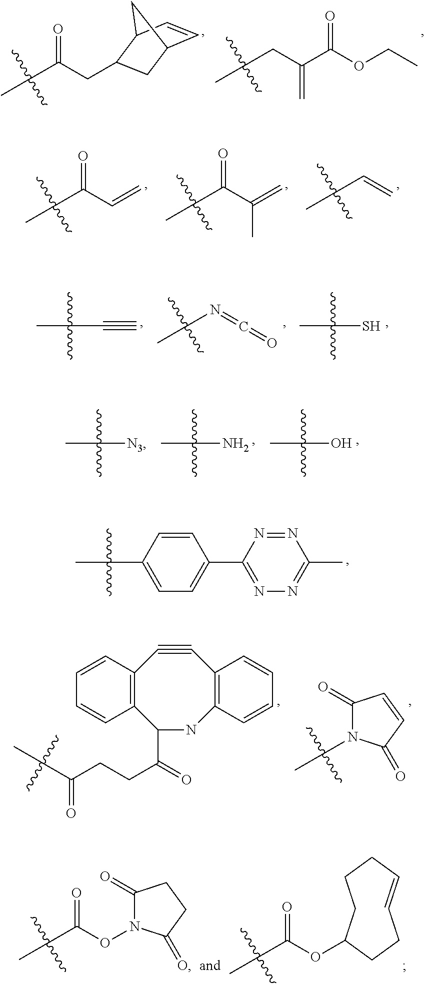

R³ is selected from the group consisting of H and methyl; and n is an integer from 1 to 500.

In certain embodiments, the encapsulating matrix material comprises at least one non-degradable crosslinker. In certain embodiments, the at least one non-degradable crosslinker is selected from the group consisting of functionalized poly(ethylene glycol), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), acrylate-functionalized gelatin, methacrylate-functionalized gelatin, acrylate-functionalized hyaluronic acid, and methacrylate-functionalized hyaluronic acid.

In certain embodiments, the at least one non-degradable crosslinker is functionalized with at least one functional moiety selected from the group consisting of acrylate, methacrylate, norbornene, thiol, azide, alkene, alkyne, oxime, hydrozone, isocyanate, tetrazine, maleimide, vinyl sulphone, dibenzocyclooctyne and NHS-ester.

In certain embodiments, the encapsulating matrix material comprises at least one degradable crosslinker as described elsewhere herein.

In certain embodiments, the polymer microspheres further comprise at least one magnetic particle. In certain embodiments, the at least one magnetic particle comprises a poly-1-lysine coating. In certain embodiments, the magnetic particle is a metal particle. In certain embodiments, the magnetic particle comprises one or more materials selected from the group consisting of ferrite, magnetite, maghemite, and gold.

In certain embodiments, the magnetic particles have a diameter of about 100 nm to about 500 nm.

In certain embodiments, the aggregation of portions of the uniformly disperse polymer microsphere composition comprises magnetically levitating the microspheres to form aggregates.

In certain embodiments, the polymer microspheres are solid microspheres.

In certain embodiments, the polymer microspheres are core-shell particles comprising an outer shell and a hollow interior.

In certain embodiments, the cells are cultured on the inner surface of the outer shell. In certain embodiments, the cells are embedded within the polymer microspheres. In certain embodiments, the cells are cultured on the surface of the polymer microspheres.

In certain embodiments, the at least one magnetic particle is attached to the cells cultured on the surface of the polymer microsphere via the poly-1-lysine coating on the at least one magnetic particle.

In certain embodiments, the polymer microspheres are monodisperse microspheres.

In certain embodiments, the polymer microspheres are fabricated through the use of a microfluidics device.

In certain embodiments, the polymer microspheres have a diameter of about 10 μm to about 300 μm. In certain embodiments, the polymer microspheres have a diameter of about 200 μm.

In certain embodiments, the polymer microspheres have a stiffness of about 1 kPa to about 100 kPa. In certain embodiments, the polymer microspheres have a stiffness of about 1 kPa to about 5 kPa or about 20 kPa to about 100 kPa.

In certain embodiments, the encapsulating matrix material has a stiffness of about 1 kPa to about 100 kPa. In certain embodiments, the encapsulating matrix material has a stiffness of about 1 kPa to about 5 kPa or about 20 kPa to about 100 kPa.

In certain embodiments, the method further comprises adjusting the stiffness of encapsulating matrix material using a dual stage curing process.

In certain embodiments, incubating the cells in the encapsulating matrix degrades the degradable crosslinkers, thereby degrading the polymer microspheres while leaving the encapsulating matrix intact.

In certain embodiments, the method further comprises testing the encapsulated cells for the presence of one or more biological markers. In certain embodiments, the one or more biological markers includes expressed RNA, expressed mRNA, expressed genes, soluble proteins, membrane-bound proteins, ECM proteins, ECM-bound proteins, cytokines, growth factors, enzymes, hormones, signaling ions, DNA content, metabolic byproducts, apoptosis markers, cell senescence markers, cell motility markers, epigenetic changes and contents of extracellular vesicles released by the cells.

In certain embodiments, the encapsulated cells are tested for the expression of one or more markers selected from the group consisting of Acta2 (α-SMA), Agt, Ccl1 (eotaxin), Ccl12 (MCP-5, Scya12), Ccl3 (Mip-1a), Ctgf, Grem1, Il13, Il13ra2, Il4, Il5, Snai1 (Snai1), Bmp7, Hgf, Ifng, Il10, Il13ra2, Col1a2, Col3a1, Lox, Mmp1a, Mmp13, Mmp14, Mmp2, Mmp3, Mmp8, Mmp9, Plat (tPA), Plau (uPA), Plg, Serpina1a, Serpine1 (PAI-1), Serpinh1 (Hsp47), Timp1, Timp2, Timp3, Timp4, Itga1, Itga2, Itga3, Itgav, Itgb1, Itgb3, Itgb5, Itgb6, Itgb8, Ccl11 (eotaxin), Ccl12 (MCP-5, Scya12), Ccl3 (Mip-1a), Ccr2, Cxcr4, Ifng, Il10, Il13, Il13ra2, Il1a, Il1b, Il4, Il5, Ilk, Tnf, Agt, Ctgf, Edni, Egf, Hgf, Pdgfa, Pdgfb, Vegfa, Bmp7, Cavl, Den, Eng (Evi-1), Grem1, Inhbe, Ltbp1, Smad2 (Madh2), Smad3(Madh3), Smad4 (Madh4), Smad6, Smad7, Tgfb1, Tgfb2, Tgfb3, Tgfbr1 (ALK5), Tgfbr2, Tgif1, Thbs1 (TSP-1), Thbs2, Cebpb, Jun, Myc, Nfkb1, Sp1, Stat1, Stat6, Akt1, Bmp7, Col1a2, Col3a1, Itgav, Itgb1, Mmp2, Mmp3, Mmp9, Serpine1 (PAI-1), Smad2 (Madh2), Snai1(Snail), Tgfb1, Tgfb2, Tgfb3, Timp1, Bcl2, and Fasl (Tnfsf6).

In certain embodiments, the encapsulating matrix material further comprises at least one type of cell selected from the group consisting of basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells, airway or bronchial epithelial cells, and cell lines comprising A549, MLE-12 and/or 3T3 fibroblasts.

In certain embodiments, the polymer microsphere composition comprises the at least one multifunctional monomer, the at least one peptide and the at least one degradable crosslinker as described elsewhere herein.

In certain embodiments, the polymer microsphere composition further comprises at least one cell. In certain embodiments, the at least one cell is selected from the group consisting of basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells, airway or bronchial epithelial cells, and cell lines comprising A549, MLE-12 and/or 3T3 fibroblasts.

In certain embodiments, the at least one multifunctional monomer, the at least one peptide, and the at least one degradable crosslinker are covalently bound to form a hydrogel.

In certain embodiments, the aggregated alveoli-like structure comprises the polymer microsphere composition of the invention.

In certain embodiments, the aggregated alveoli-like structure include the polymer microspheres encapsulated in the encapsulating matrix, as outlined elsewhere herein, comprising the at least one multifunctional monomer, the at least one crosslinker; and the at least one peptide segment. In certain embodiments, the at least one crosslinker in the encapsulating matrix is different from the at least one degradable crosslinker in the polymer microsphere composition.

In certain embodiments, the encapsulating matrix comprises the at least one peptide segment comprising CGRGDS. In certain embodiments, the encapsulating matrix comprises the at least one peptide segment comprising CGYIGSR.

In certain embodiment, the encapsulating structure further comprises a peptide segment comprising CGRGDS. In certain embodiment, the encapsulating structure further comprises a peptide segment comprising CGYIGSR. In certain embodiments, the at least one peptide segment in the polymer microspheres and the at least one peptide segment in the encapsulating matrix are independently selected and may be either the same or different.

In certain embodiment, the structure has a stiffness of about 1 kPa to about 100 kPa.

In certain embodiments, the subject being treated is in need thereof.

In certain embodiments, the composition further comprises at least one pharmaceutical agent, growth factor, cytokine, or any other biochemical agent.

In certain embodiments, the subject is further administered one or more gene therapies.

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIG. 1A is an image of a pair of human lungs, showing the network of airway branches terminating with spherical alveoli (inset image), each having a diameter of about 200 μm.

FIGS. 1B-1C are images of degradable microspheres according to an embodiment of the invention, seeded with primary lung cells and embedded in a hydrogel matrix that mimics the ECM environment of a lung in stiffness and composition. As the cells grow, they naturally secrete enzymes that can degrade the microsphere templates, resulting in structures that replicate alveoli.

FIG. 2 is a scheme showing a general chemical structure of an exemplary composition of the invention. Polyethylene glycol norbornene (PEG-NB) (top) reacts with an MMP-degradable crosslinker (second from top) and a peptide sequence mimicking fibronectin (bottom) to create hydrogel microsphere templates. PEG-NB (top) also reacts with a non-degradable PEG-dithiol (second from bottom) and synthetic peptide mimics (bottom) of critical basement membrane components found in lung to form hydrogel matrices tailored to mimic fibrotic lung for encapsulation of cell-coated microsphere templates.

FIGS. 3A-3D are schemes, images, and graphs showing hydrogel microspheres synthesized using inverse suspension polymerization, microsphere mechanical properties, size distribution of filtered hydrogel microspheres, and degradation of hydrogel microspheres by collagenase I. FIG. 3A is a scheme of microsphere synthesis through inverse suspension method. FIG. 3B displays the Young's modulus of microsphere hydrogel made from (PEG-NB) with an MMP-degradable crosslinker. FIG. 3C shows the microsphere distribution with a mean of 198.5±82.4 m in diameter which mimicks alveolar structure (d˜200 μm). FIG. 3D shows the degradation of microspheres in varying concentrations of collagenase I by bead size (top), average intensity within the microsphere (middle), and average intensity outside the microsphere (bottom).

FIG. 4A is a scheme depicting an exemplary method of fabricating the microspheres of the invention using PEG-NB and an MMP degradable crosslinker. An aqueous solution of PEG-NB, MMP-degradable crosslinker, peptide and a photoinitiator (LAP) flow into one arm of a t-junction, while an organic phase (Tween 20 and Span 80 in hexane) flows into the other arm to form microspheres. Microspheres created in the microfluidic devices are collected in a bath with the same composition as the organic phase and polymerized by exposure to UV light.

FIG. 4B is a scheme depicting an exemplary method of fabricating a hydrogel core-shell microparticle of the invention using PEG-NB and an MMP degradable crosslinker through the use of a microfluidics device. In certain embodiments, an ageous solution of PEG-NB, MMP-degradable crosslinker, peptide, and a photoinitiator enters the microfluidic device as the shell phase. A second aqueous solution of either culture media or PBS enters the microfluidic device as the core phase. A hydrophobic suspension enters the microfluidic device as the oil phase. Precision in microfluidic design and phase flow rate allows specific control of phase mixing, particle size, and shell thickness. Cells may be incorporated in either (a) the core phase or (b) the shell phase, or (c) may be seeded on the particle surface following particle fabrication.

FIGS. 5A-5B are schemes showing exemplary cell templating procedures of the invention. Primary ATII cells are seeded onto biodegradable microsphere templates by exposing cells to microspheres suspended in sterile cell culture media in an ultra-low adhesion 24-well plate. Following incubation microspheres aggregate into clusters to recapitulate 3D alveolar structure. FIG. 5B further shows the degradation of the degradable crosslinkers, leaving behind 3D cellular structures in the shape of the aggregated microspheres.

FIG. 6 is a graph reporting the stiffness of 10 kg/mol PEG-NB compositions vs. 40 kg/mol PEG-NB compositions and how they compare to healthy and fibrotic lung tissue. This graph shows that PEG-based hydrogels can be tailored to mimic stiffness values of both healthy and fibrotic lung tissue.

FIG. 7 is a scheme showing a representative crosslink network of a spatiotemporally addressable, hydrolytically stable hydrogel material, according to an embodiment of the invention. Off-stoichiometric thiol-ene chemistry enables spatiotemporal crosslinking and elevation of local elastic modulus via visible-light irradiation. The nitrobenzyl-ether derivative within the dithiol crosslinker facilitates UV photolysis and reduction of local elastic modulus. Exclusive use of unique alpha-methacrylate and sulfonate ester functionalities mitigate bulk network hydrolysis. These properties result in hydrolytically stable hydrogel networks with moduli that can be amplified or reduced with both spatial and temporal resolution.

FIG. 8 is a scheme showing a representative crosslink network of a spatiotemporally addressable, hydrolytically stable hydrogel material, according to an embodiment of the invention. Off-stoichiometric thiol-ene chemistry enables spatiotemporal crosslinking and elevation of local elastic modulus via visible-light irradiation. The nitrobenzyl-ether derivative within the PEG backbone facilitates UV photolysis and reduction of local elastic modulus. Exclusive use of unique alpha-methacrylate functionalities mitigate bulk network hydrolysis. These properties result in hydrolytically stable hydrogel networks with moduli that can be amplified or reduced with both spatial and temporal resolution.

FIG. 9 is a scheme comparing organoid culture techniques. The top path illustrates traditional culture techniques relying on animal-derived matrices which exhibit high levels of heterogeneity and inconsistency. The middle path illustrates culture techniques utilizing synthetic matrices, either known in the art or of the invention, having varies stiffnesses. The bottom path illustrates culture techniques utilizing the matrices of the invention that are precisely tunable, and capable of facilitating well-defined, complete differentiation of human pulmonary epithelium from iPSCs. The bottom path shows in Step 4 that the matrices of the invention allow for spatiotemporal control over initiation of a profibrotic phenotype in encapsulated epithelial cells and fibroblasts to improve in vitro models of fibrosis.

FIG. 10A is a scheme of nanoshuttle coated epithelial cell microsphere coating and subsequent aggregation through a magnetic drive.

FIG. 10B is a graph of aggregate size dependence on theoretical (dashed lines) and experimental (square points) microsphere size.

FIG. 10C is aggregate images at different cell seeding densities. Sectioned fluorescent images contain microspheres, actin, DAPI (Scale bar=100 μm).

FIG. 10D is confocal images showing that the microspheres were aggregated into structures that mimic distal lung tissue geometries when seeded with A594 cells (actin, red; DAPI, blue) (Scale bar=100 μm).

FIG. 10E is a graph showing viability measured by a WST-1 assay indicating no significant deviation over 14 days.

FIG. 11A-11B are reaction schematic of base-catalyzed step growth or radical polymerized chain growth of alpha-methacrylate (aMA) and methacrylate (MA). Hydrolysis at the ester moiety does not impact integrity of the parent polymer chain and results in minute quantities of ethanol.

FIGS. 12A-12B are graphs demonstrating the photocontrol of elastic modulus elevation in aMA hydrogel materials of the invention. FIG. 12A is a graph showing a maximum of ˜2 fold increase in elastic modulus upon light exposure and control of extend of modulus change with tuned off-stoichiometric ratios of the hydrogel, as measured by static rheology. FIG. 12B is a graph showing that phototunable materials can be fabricated with an elastic modulus in the range of healthy lung tissue and then stiffened dynamically through exposure to photoexcitation, as measured by rheology. FIG. 12B shows two distinct stages of elastic modulus evolution during the initial base-catalyzed gelation followed by a photo-controlled chain growth step, as determined by in situ rheology. Elevation of elastic modulus was more pronounced prior to swelling due to higher proximity of reactive groups within the parent network.

FIG. 13A is a graph of the Young's modulus over time of both PEGαMA and PEGMA hydrogels showing that PEGαMA hydrogels resist hydrolysis compared to traditional PEGMA chemistries.

FIG. 13B images demonstrating the spatial control over the stiffening reaction by using photomasks the material can be spatially patterned as visualize by an Alexa 555 tagged vinyl group.

FIG. 13C shows the dual cure system developed here enables us to embed cells in initially soft hydrogels that mimic healthy tissue and stiffen to emulate fibrotic progression.

FIG. 13D is a graph of the Young's modulus of hydrogel formulations.

FIG. 13E-13G are graphs of the normalized YAP intensity, circularity, and aspect ratio of A549 cells on tissue culture plastic (control) verses soft and stiff hydrogel formulations demonstrating that the material mechanics has an effect on the YAP activation pathway.

FIG. 14 is a scheme showing the application of hydrolytically-resistant, spatiotemporally addressable hydrogels within a 3D in vitro model of IPF, according to an embodiment of the invention.

FIGS. 15A-15B are schematics and graphs of flow sorted epithelial cells and fibroblasts from dual-reporter mice templated into biomaterial system to emulate fibrosis in vitro.

### DETAILED DESCRIPTION OF THE INVENTION

In one aspect, the present invention relates to the unexpected discovery of tissue mimicking constructs and compositions that can be used to study growth and development of cells in vitro. In certain embodiments, the invention provides methods of culturing cells on the tissue mimicking compositions of the invention. In other embodiments, the invention provides methods of treating a disease or disorder using the compositions and constructs of the invention.

### Definitions

As used herein, each of the following terms has the meaning associated with it in this section.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

Generally, the nomenclature used herein and the laboratory procedures in tissue engineering and biomaterial science are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of 20% or 10%, more preferably +5%, even more preferably 1%, and still more preferably 0.1% from the specified value, as such variations are appropriate to perform the disclosed methods.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

As used herein, the terms “covalently bound” or “covalently conjugated” refers to the formation of a covalent bond between two chemical species or moieties. Covalent bonds are to be taken to have the meaning commonly accepted in the art, referring to a chemical bond that involves the sharing of electron pairs between atoms.

As used herein “crosslinking” is meant to be a process of creating a bond that links one polymer chain to another. Crosslinking bonds are often in the form of covalent bonds or ionic bonds, however in some instances crosslinking can take place through non-covalent interactions, such as but not limited to hydrogen bonds, pi stacking interactions or metal-ligand coordination.

As used herein “crosslinking agent” or “crosslinking source” is meant to be an agent that is capable of forming a chemical or ionic links between molecules.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “gel” refers to a three-dimensional (3D or 3-D) polymeric structure that itself is insoluble in a particular liquid but that is capable of absorbing and retaining large quantities of the liquid to form a stable, often soft and pliable, but always to one degree or another shape-retentive, structure. When the liquid is water, the gel is referred to as a hydrogel. Unless expressly stated otherwise, the term “gel” is used throughout this application to refer both to polymeric structures that have absorbed a liquid other than water and to polymeric structures that have absorbed water, it being readily apparent to those skilled in the art from the context whether the polymeric structure is simply a “gel” or a “hydrogel.”

As used herein, the term “microsphere” refers to a spherical or spheroid particle with a diameter in the range of about 1 μm to about 1 mm. In certain embodiments, microspheres comprise one or more layers, optionally including an outer shell layer, while in other embodiments, microspheres do not comprise layers or an outer shell.

As used herein, the term “monodisperse” refers to a particle based composition comprising particles that are substantially uniform in size, shape and mass. In certain embodiments, a monodisperse composition of microspheres contains particles of nearly the same size, forming a narrow distribution about an average value, whereas a polydisperse suspension contains particles of different sizes, forming a broad distribution.

In certain embodiments, monodisperse or near-monodisperse particles have equal to or less than about 15% coefficient of variation. In other embodiments, monodisperse particles have equal to or less than about 5% coefficient of variation (that is, CV=σ/d<5%, where σ and d are the standard deviation and the mean size, respectively). In yet other embodiments, the monodisperse particles have equal to or less than about 5%, 2%, or 1%.

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent, growth factor, cytokine, or any other biochemical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following abbreviations are used herein: aMA, alpha-methacrylate; CTGF, connective tissue growth factor; EDBE, 2,2′-(ethylenedioxy)bis(ethylamine); ECM, extracellular matrix; IPF, idiopathic pulmonary fibrosis; iPSC, induced-pulipotent stem cell MMP, matrix metalloprotease; PCL, poly-caprolactone; PCL-PLGA, copolymers of polylactic-glycolic acid and poly-caprolactone; PCR, polymerase chain reaction; PDGF, platelet-derived growth factor; PEG, polyethylene glycol; PEO-PBTP, polyethylene oxide-butylene terephthalate; PLA-DX-PEG, poly-D,L-lactic acid-p-dioxanone-polyethylene glycol block copolymer; PLGA, polylactic-glycolic acid; POE, polyorthoester; SFTPB, surfactant protein B; SFTPC, surfactant protein C.

### Compositions

In one aspect, the invention provides polymer microsphere compositions and constructs for use in treating lung diseases and disorders and/or in testing methods of treating lung diseases and disorder. In certain embodiments, the invention provides compositions and constructs useful for treating or testing methods of treating lung diseases and disorders selected from, but not necessarily limited to, pulmonary fibrosis, chronic obstructive pulmonary diseases (COPD) including emphysema, chronic bronchitis, refractory asthma and bronchiectasis, cancer, pulmonary hypertension, and cystic fibrosis.

In certain embodiments, the invention includes a polymer microsphere composition comprising at least one multifunctional monomer; at least one peptide segment from at least one protein; and at least one degradable crosslinker. In certain embodiments, the polymer microspheres further comprise at least one cell.

In certain embodiments, the at least one multifunctional monomer is selected from the group consisting of functionalized poly(ethylene glycol), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), acrylate- and methacrylate functionalized natural polymers such as gelatin or hyaluronic acid. In other embodiments, the at least one multifunctional monomer is functionalized with at least one functional moiety selected from the group consisting of acrylate, methacrylate, norbornene, thiol, azide, alkene, alkyne, oxime, hydrozone, isocyanate, tetrazine, maleimide, vinyl sulphone, dibenzocyclooctyne, and NHS-ester. In yet other embodiments, the at least one multifunctional monomer is functionalized with at least two, at least three, at least four or at least eight functional groups.

In certain embodiments, the at least one multifunctional monomer is a compound of Formula (I):

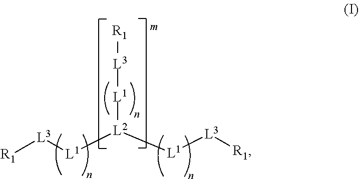

wherein:

each instance of L¹ is independently a polymeric linker moiety comprising at least one selected from the group consisting of polyethylene glycol (PEG), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), and poly-D,L-lactic acid-p-dioxanone-polyethylene glycol block copolymer (PLA-DX-PEG);

L² is a polymeric linker moiety comprising at least one selected from the group consisting of polyglycerol, and polypentaerythritol;

each instance of L³ independently comprises at least one linkage selected from the group consisting of a bond, an ether linkage, an ester linkage, a sulfonate ester linkage and an amide linkage;

each instance of R¹ independently comprises a functionality selected from the group consisting of acrylate, methacrylate, norbornene, thiol, tetrazine, amine, dibenzocyclooctyne, maleimide, succinimide, trans-cyclooctene, azide, alkene, alkyne, oxime, hydrazone, alcohol, and isocyanate;

m is an integer from 0 to 10; and n is an integer from 1 to 500.

In certain embodiments, the at least one multifunctional monomer is a compound of Formula (IA):

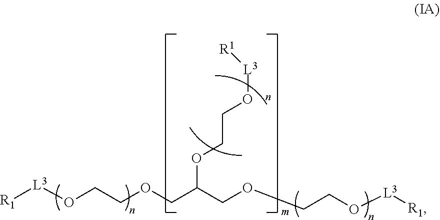

wherein:

each instance of L³ independently comprises at least one linkage selected from the group consisting of a bond, an ether linkage, an ester linkage, a sulfonate ester linkage and an amide linkage;

each instance of R¹ independently comprises a functionality selected from the group consisting of acrylate, methacrylate, norbornene, thiol, tetrazine, amine, dibenzocyclooctyne, maleimide, succinimide, trans-cyclooctene, azide, alkene, alkyne, oxime, hydrazone, alcohol, and isocyanate;

m is an integer from 0 to 10; and n is an integer from 1 to 500.

In certain embodiments, m is an integer from 0 to essentially any integer desired. In other embodiments, m is larger than 10 and can be determined by a person of ordinary skill in the art based on the desired qualities of the resulting composition. In yet other embodiments, m is 2. In yet other embodiments, m is 6.

In certain embodiments, n is an integer from 1 to essentially any integer desired. In other embodiments, n is larger than 500 and can be determined by a person of ordinary skill in the art based on the desired qualities of the resulting composition. In yet other embodiments, n is 114. In yet other embodiments, n is 454.

In certain embodiments, L³ is a bond or a linkage having a structure selected from the group consisting of:

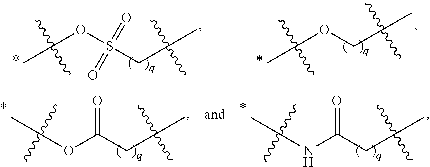

wherein the * side of the linkage is bound to the monomer and the opposite side is bound to R¹, and wherein q is an integer selected from 0 to 6.

In certain embodiments, R¹ is a functionality having a structure selected from the group consisting of:

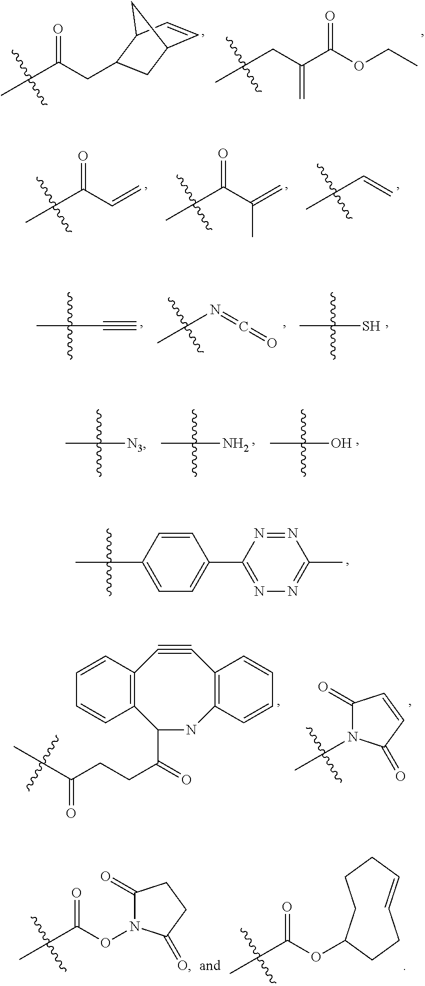

In certain embodiments, the multifunctional monomer is functionalized with functional groups that can participate in one or more “click-chemistry” reactions with the at least one degradable crosslinker. In other embodiments, the “click-chemistry” reaction is selected from, but not necessarily limited to, azide-alkyne cycloaddition, thiol-vinyl addition, thiol-yne, thiol-isocyanate, Michael addition, 1,3 diploar cycloaddition, Diels-Alder addition and oxime/hydrazine formation.

In certain embodiments, the at least one peptide segment is a segment from at least one protein selected from the group consisting of matrisome proteins and matrisome-associated proteins. In certain embodiments, the matrisome proteins comprise glycoproteins, proteoglycans and collagen. In certain embodiments, the matrisome-associated proteins comprise secreted factors, extracellular matrix-affiliated proteins and extracellular matrix regulators. In certain embodiments, the at least one peptide segment is at least one segment of at least one protein selected from the group consisting of collagen, elastin, fibronectin, laminin, fibrillin, tenascin, vitronectin, serpin, asporin, and osteonectin. In other embodiments, the at least one peptide segment is a synthetic peptide segment that mimics a segment of at least one protein selected from the group consisting of collagen, elastin, fibronectin, laminin, fibrillin, tenascin, serpin, asporin, vitronectin, and osteonectin. In yet other embodiments, the at least one peptide segment comprises CGRGDS. In yet other embodiments, the at least one peptide segment comprises CGYIGSR. In yet other embodiments, an additional peptide segment comprising CGRGDS is present along with the at least one peptide segment. In yet other embodiments, an additional peptide segment comprising CGYIGSR is present along with the at least one peptide segment.

In certain embodiments, the at least one cell is selected from the group consisting of basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells airway or bronchial epithelial cells and cell lines comprising A549, MLE-12 and/or 3T3 fibroblasts.

In certain embodiments, the composition comprises at least two types of cells. In other embodiments, the composition comprises at least two types of cells arranged in layers such that each layer comprises a different type of cell.

In certain embodiments, the at least one degradable crosslinker is an enzyme-degradable crosslinker, a protease-degradable crosslinker, a photodegradable crosslinker or a biodegradable crosslinker. In other embodiments, the at least one degradable crosslinker is a matrix metalloprotease (MMP) degradable crosslinker. In yet other embodiments, the at least one degradable crosslinker is a crosslinker that can be degraded in the presence of photoexcitation. In yet other embodiments, the photoexcitation is visible light photoexcitation (380 nm-760 nm) or ultraviolet (UV) light photoexcitation (100 nm-380 nm). In yet other embodiments, the at least one degradable crosslinker comprises at least one selected from the group consisting of ortho-nitrobenzyl moieties, coumarin, azobenzene, rotaxane, dithiols, aromatic disulfides, poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), spermine, 2,2′-(ethylenedioxy)bis(ethylamine) (EDBE), CGPQGIWGQGC peptide, GPQGIAGQ peptide (PCL-1) and IPVSLRSG peptide (PCL-2).

In certain embodiments, the at least one degradable crosslinker is at least one compound selected from the group consisting of CGPQGIWGQGC peptide, GPQGIAGQ peptide (PCL-1), and IPVSLRSG peptide (PCL-2).

In certain embodiments, the at least one degradable crosslinker is functionalized with at least two, at least three, at least four or at least eight functional groups.

In certain embodiments, the at least one degradable crosslinker is a compound of Formula (II):

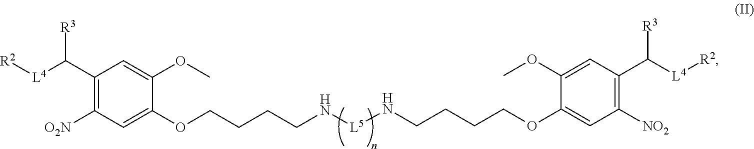

wherein:

each instance of L⁴ independently comprises at least one linkage selected from the group consisting of a sulfonate ester linkage and an amide linkage;

L⁵ is a polymeric linker moiety comprising at least one selected from the group consisting of polyethylene glycol (PEG), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), and poly-D,L-lactic acid-p-dioxanone-polyethylene glycol block copolymer (PLA-DX-PEG);

each instance of R² independently comprises a functionality selected from the group consisting of acrylate, methacrylate, norbornene, thiol, tetrazine, amine, dibenzocyclooctyne, maleimide, succinimide, trans-cyclooctene, azide, alkene, alkyne, oxime, hydrazone, alcohol, and isocyanate;

R³ is selected from the group consisting of H and methyl; and

n is an integer from 1 to 500.

In certain embodiments, n is an integer from 1 to essentially any integer desired. In other embodiments, n is larger than 500 and can be determined by a person of ordinary skill in the art based on the desired qualities of the resulting composition. In yet other embodiments, n is 114. In yet other embodiments, n is 454.

In certain embodiments, L⁴ is a linkage having a structure selected from the group consisting of:

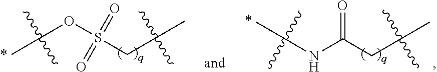

wherein the * side of the linkage is bound to the monomer and the opposite side is bound to R², and wherein q is an integer selected from 0 to 6.

In certain embodiments, R² is a functionality having a structure selected from the group consisting of:

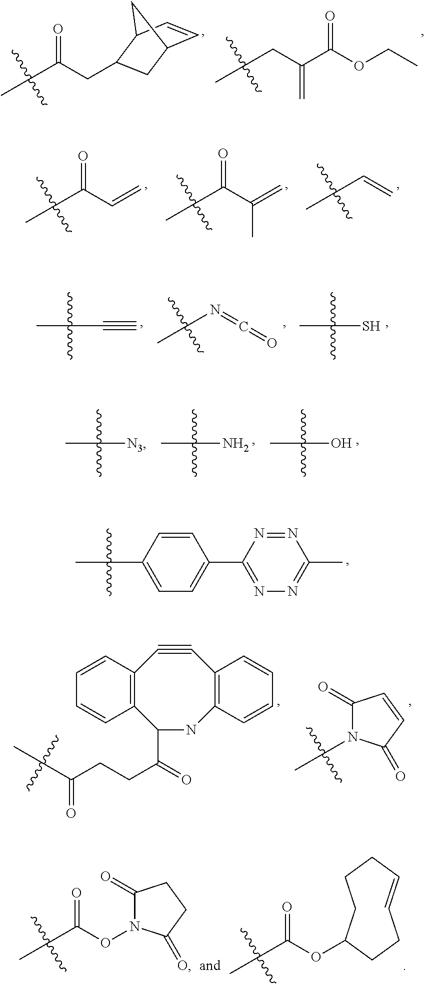

In certain embodiments, the polymer microspheres are solid microspheres comprising a single continuous sphere of polymer without any internal voids or cavities. In other embodiments, the polymer microspheres are core-shell particles comprising an outer shell and a hollow interior. In certain embodiments, the polymer microsphere composition comprises microspheres that are substantially uniform. In other embodiments, the composition is a monodisperse microsphere composition wherein the microspheres in the composition have a coefficient of variation (CV) of less than about 15% from one another. In certain embodiments, the microspheres are fabricated through the use of a microfluidics device. Without wishing to be limited to any particular theory, the use of a microfluidics device in fabricating the microspheres can yield a monodisperse microsphere composition.

In certain embodiments, the at least one cell is imbedded in the polymer microspheres. In other embodiments, the at least one cell is on the surface of the polymer microspheres. In yet other embodiments wherein the polymer microspheres are core-shell particles, the at least one cell is imbedded on the interior surface of the core-shell particle. In other embodiments, the at least one cell is on the surface of the polymer core-shell particles.

In certain embodiments, the polymer microsphere composition comprises the multifunctional monomer and the degradable crosslinker in amounts such that the molar ratio of multifunctional monomer functional groups to degradable crosslinker functional groups is greater than about 1:1, about 1.5:1, about 8:5, about 2:1, about 8:3 or greater than about 8:3. In yet other embodiments, the composition comprises more multifunctional monomer functional groups than degradable crosslinker functional groups.

In certain embodiments, the polymer microspheres are core-shell microspheres.

In certain embodiments, the polymer microspheres further comprise at least one magnetic particle. In certain embodiments, the magnetic particle is a metal particle. In other embodiments, the magnetic particle comprises one or more materials selected from the group consisting of ferrite, magnetite, maghemite, and gold. In certain embodiments, the magnetic particles have a diameter of about 100 nm to about 500 nm. In certain embodiments, the magnetic particle comprises a poly-1-lysine coating.

In certain embodiments, the magnetic particle is attached to the cells cultured on the surface of the polymer microsphere via the poly-1-lysine coating on the magnetic particle.

In certain embodiments, the at least one multifunctional monomer, at least one peptide and at least one degradable crosslinker are covalently bound to form a hydrogel. In other embodiments, the hydrogel comprises covalent bonds between the at least one multifunctional monomers and the at least one degradable crosslinker. In yet other embodiments, the hydrogel comprises covalent bonds between at least two of the at least one multifunctional monomers. In yet other embodiments, the hydrogel comprises more multifunctional monomer functional groups than degradable crosslinker functional groups, such that at least a portion of multifunctional monomer functional groups are covalently bound to degradable crosslinker functional groups and at least a separate portion of multifunctional monomer functional groups are covalently bound to other multifunctional monomer functional groups.

In certain embodiments, the polymer microspheres have a diameter of about 10 μm to about 300 μm. In other embodiments, the polymer microspheres have a diameter of about 200 μm. In certain embodiments, the polymer microspheres have a stiffness of about 1 kPa to about 100 kPa. In other embodiments, the polymer microspheres have a stiffness of about 1 kPa to about 5 kPa or about 20 kPa to about 100 kPa. In certain embodiments, the stiffness of the polymer microspheres can be adjusted by altering the ratio of the multifunctional monomer and the degradable crosslinker or by changing the identity of either species, including but not limited to increasing of decreasing the number of recpeating units or molecular weight of either species. In other embodiments, the stiffness of the polymer microspheres can be adjusted by altering the water content of the composition. In other embodiments, the stiffness of the polymer microspheres can be adjusted by exposing the microspheres to photoexcitation, whereby the photoexcitation increases induces additional crosslinking in the polymer microspheres. In yet other embodiments, the photoexcitation induces crosslinking of at least a portion of multifunctional monomer functional groups with other multifunctional monomer functional groups. In yet other embodiments, the stiffness of the microspheres can be adjusted by exposing the microspheres to photoexcitation, whereby the photoexcitation degrades at least a portion of the degradable crosslinker in the polymer microspheres. In yet other embodiments, the photoexcitation can be localized photoexcitation, allowing for spatiotemporal control of the stiffness of the polymer microspheres.

In certain embodiments, the polymer microspheres are hydrolytically stable, in that they are resistant to hydrolysis.

The invention further provides aggregated microsphere structures comprising the polymer microspheres of the invention. In certain embodiments, the aggregates are alveoli-like structures that closely mimic the structure and shape of the alveoli of a mammalian lung.

In certain embodiments, the aggregates comprise the polymer microsphere composition of the invention encapsulated within a matrix comprising at least one multifunctional monomer; at least one crosslinker; and at least one peptide segment from at least one protein. In certain embodiments, the at least one crosslinker is a non-degradable crosslinker. In other embodiments, the at least one crosslinker is a degradable crosslinker. In other embodiments, the matrix further comprises at least one type of cell.

In certain embodiments, the at least one multifunctional monomer is selected from the group consisting of functionalized poly(ethylene glycol), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), acrylate- and methacrylate functionalized natural polymers such as gelatin or hyaluronic acid. In other embodiments, the at least one multifunctional monomer is functionalized with at least one functional moiety selected from the group consisting of acrylate, methacrylate, norbornene, thiol, azide, alkene, alkyne, oxime, hydrozone, isocyanate, tetrazine, maleimide, vinyl sulphone, dibenzocyclooctyne, and NHS-ester. In yet other embodiments, the at least one multifunctional monomer is functionalized with at least two, at least three, at least four or at least eight functional groups. In certain embodiments, the at least one multifunctional monomers in the polymer microspheres and the matrix are independently selected and may be either the same or different.

In certain embodiments, the at least one multifunctional monomer is a compound of Formula (I):

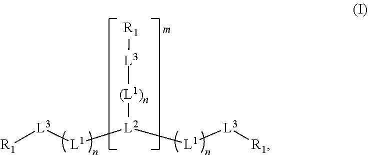

wherein:

each instance of L¹ is independently a polymeric linker moiety comprising at least one selected from the group consisting of polyethylene glycol (PEG), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), and poly-D,L-lactic acid-p-dioxanone-polyethylene glycol block copolymer (PLA-DX-PEG);

L² is a polymeric linker moiety comprising at least one selected from the group consisting of polyglycerol, and polypentaerythritol;

each instance of L³ independently comprises at least one linkage selected from the group consisting of a bond, an ether linkage, an ester linkage, a sulfonate ester linkage and an amide linkage;

each instance of R¹ independently comprises a functionality selected from the group consisting of acrylate, methacrylate, norbornene, thiol, tetrazine, amine, dibenzocyclooctyne, maleimide, succinimide, trans-cyclooctene, azide, alkene, alkyne, oxime, hydrazone, alcohol, and isocyanate;

m is an integer from 0 to 10; and n is an integer from 1 to 500.

In certain embodiments, the at least one multifunctional monomer is a compound of Formula (IA):

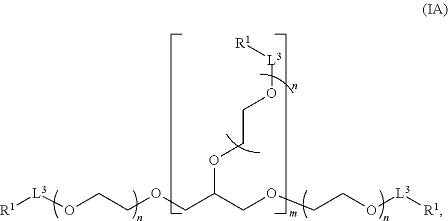

wherein:

each instance of L³ independently comprises at least one linkage selected from the group consisting of a bond, an ether linkage, an ester linkage, a sulfonate ester linkage and an amide linkage;

each instance of R¹ independently comprises a functionality selected from the group consisting of acrylate, methacrylate, norbornene, thiol, tetrazine, amine, dibenzocyclooctyne, maleimide, succinimide, trans-cyclooctene, azide, alkene, alkyne, oxime, hydrazone, alcohol, and isocyanate;

m is an integer from 0 to 10; and n is an integer from 1 to 500.

In certain embodiments, m is an integer from 0 to essentially any integer desired. In other embodiments, m is larger than 10 and can be determined by a person of ordinary skill in the art based on the desired qualities of the resulting composition. In yet other embodiments, m is 2. In yet other embodiments, m is 6.

In certain embodiments, n is an integer from 1 to essentially any integer desired. In other embodiments, n is larger than 500 and can be determined by a person of ordinary skill in the art based on the desired qualities of the resulting composition. In yet other embodiments, n is 114. In yet other embodiments, n is 454.

In certain embodiments, L³ is a bond or a linkage having a structure selected from the group consisting of:

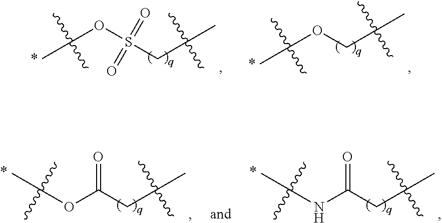

wherein the * side of the linkage is bound to the monomer and the opposite side is bound to R¹, and wherein q is an integer selected from 0 to 6.

In certain embodiments, R¹ is a functionality having a structure selected from the group consisting of:

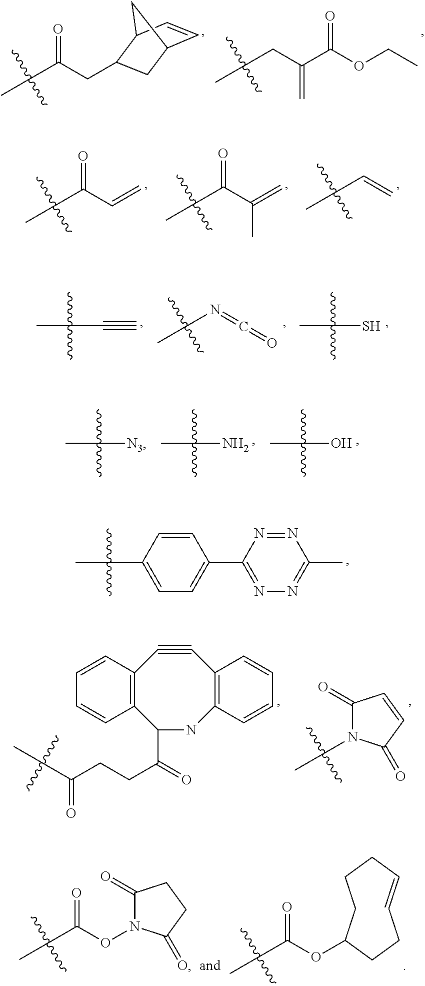

In certain embodiments, the multifunctional monomer is functionalized with functional groups that can participate in one or more “click-chemistry” reactions with the at least one degradable crosslinker. In other embodiments, the “click-chemistry” reaction is selected from, but not necessarily limited to, azide-alkyne cycloaddition, thiol-vinyl addition, thiol-yne, thiol-isocyanate, Michael addition, 1,3 diploar cycloaddition, Diels-Alder addition and oxime/hydrazine formation.

In certain embodiments, the multifunctional monomer in the encapsulating matrix is different from the multifunctional monomer in the polymer microsphere composition.

In certain embodiments, the at least one peptide segment is at least one segment of at least one protein selected from the group consisting of collagen, elastin, fibronectin, laminin, fibrillin, tenascin, vitronectin, serpin, asporin, and osteonectin.

In certain embodiments, the at least one non-degradable crosslinker is selected from the group consisting of functionalized poly(ethylene glycol), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), acrylate- and methyacrylate functionalized natural polymers such as gelatin or hyaluronic acid. In other embodiments, the at least one non-degradable crosslinker is functionalized with at least one functional moiety selected from the group consisting of acrylate, methacrylate, norbornene, thiol, azide, alkene, alkyne, oxime, hydrozone, isocyanate, tetrazine, maleimide, vinyl sulphone, dibenzocyclooctyne and NHS-ester. In yet other embodiments, the at least one non-degradable crosslinker is dithiothreitol.

In certain embodiments, the at least one non-degradable crosslinker is functionalized with at least two, at least three, at least four or at least eight functional groups.

In certain embodiments, the at least one degradable crosslinker is an enzyme-degradable crosslinker, a protease-degradable crosslinker, a photodegradable crosslinker or a biodegradable crosslinker. In other embodiments, the at least one degradable crosslinker is a matrix metalloprotease (MMP) degradable crosslinker. In yet other embodiments, the at least one degradable crosslinker comprises at least one selected from the group consisting of ortho-nitrobenzyl moieties, coumarin, azobenzene, rotaxane, aromatic disulfides, dithiols, poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), spermine, 2,2′-(ethylenedioxy)bis(ethylamine) (EDBE), CGPQGIWGQGC peptide, GPQGIAGQ peptide (PCL-1) and IPVSLRSG peptide (PCL-2). In certain embodiments, the at least one degradable crosslinker is at least one compound selected from the group consisting of CGPQGIWGQGC peptide, GPQGIAGQ peptide (PCL-1), and IPVSLRSG peptide (PCL-2).

In certain embodiments, the at least one degradable crosslinker is functionalized with at least two, at least three, at least four or at least eight functional groups.

In certain embodiments, the at least one degradable crosslinker is a compound of Formula (II):

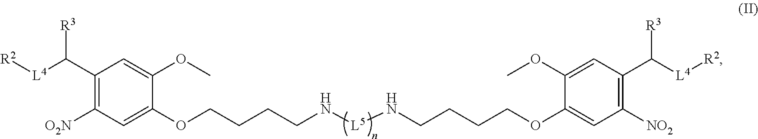

wherein:

each instance of L⁴ independently comprises at least one linkage selected from the group consisting of a sulfonate ester linkage and an amide linkage;

L⁵ is a polymeric linker moiety comprising at least one selected from the group consisting of polyethylene glycol (PEG), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), and poly-D,L-lactic acid-p-dioxanone-polyethylene glycol block copolymer (PLA-DX-PEG);

each instance of R² independently comprises a functionality selected from the group consisting of acrylate, methacrylate, norbornene, thiol, tetrazine, amine, dibenzocyclooctyne, maleimide, succinimide, trans-cyclooctene, azide, alkene, alkyne, oxime, hydrazone, alcohol, and isocyanate;

R³ is selected from the group consisting of H and methyl; and

n is an integer from 1 to 500.

In certain embodiments, n is an integer from 1 to essentially any integer desired. In other embodiments, n is larger than 500 and can be determined by a person of ordinary skill in the art based on the desired qualities of the resulting composition. In yet other embodiments, n is 114. In yet other embodiments, n is 454.

In certain embodiments, L⁴ is a linkage having a structure selected from the group consisting of:

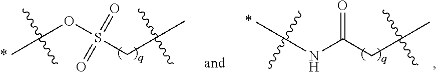

wherein the * side of the linkage is bound to the monomer and the opposite side is bound to R², and wherein q is an integer selected from 0 to 6.

In certain embodiments, R² is a functionality having a structure selected from the group consisting of:

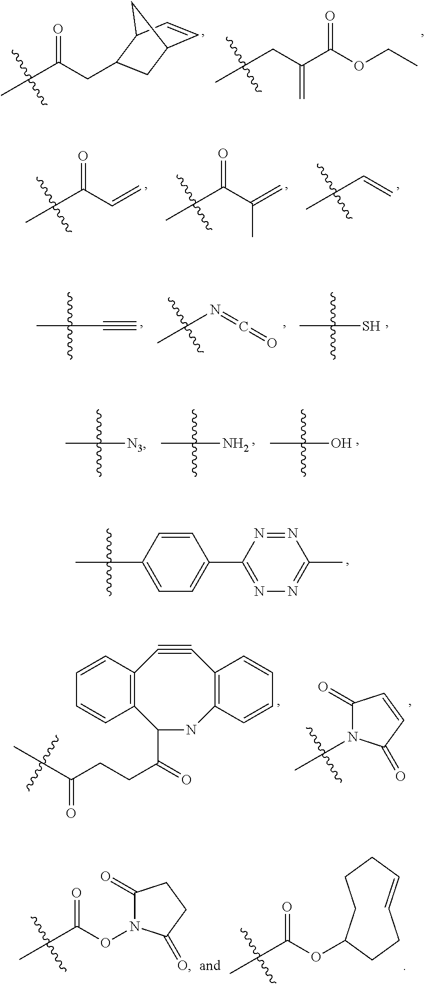

In certain embodiments, the degradable crosslinker in the encapsulating matrix is different from the degradable crosslinker in the polymer microsphere composition.

In certain embodiments, the crosslinker in the encapsulating matrix material is same as the degradable crosslinker in the polymer microsphere composition.

In certain embodiments, the encapsulating matrix comprises the multifunctional monomer and the crosslinker in amounts such that the molar ratio of multifunctional monomer functional groups to crosslinker functional groups is greater than about 1:1, about 1.5:1, about 8:5, about 2:1, about 8:3 or greater than about 8:3. In yet other embodiments, the encapsulating matrix comprises more multifunctional monomer functional groups than crosslinker functional groups. In certain embodiments, the encapsulating matrix comprises at least one cell selected from the group consisting of basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells, airway or bronchial epithelial cells and cell lines comprising A549, MLE-12 and/or 3T3 fibroblasts

In certain embodiments, the microspheres are aggregated together through the use of magnetic forces which influence magnetic particles embedded within the polymer microspheres. In other embodiments, the microspheres are aggregated together through the use of a microwell template. In certain embodiments, the alveoli-like structures have a stiffness of about 1 kPa to about 100 kPa. In other embodiments, the alveoli-like structures have a stiffness of about 1 kPa to about 5 kPa or about 20 kPa to about 100 kPa. In certain embodiments, the stiffness of the alveoli-like structures can be adjusted by altering the ratio of the multifunctional monomer and the non-degradable crosslinker or by changing the identity of either species.

In certain embodiments, the at least one multifunctional monomer, at least one peptide and at least one crosslinker are covalently bound to form a hydrogel. In other embodiments, the hydrogel comprises covalent bonds between the at least one multifunctional monomers and the at least one crosslinker. In yet other embodiments, the hydrogel comprises covalent bonds between at least two of the at least one multifunctional monomers. In yet other embodiments, the hydrogel comprises more multifunctional monomer functional groups than crosslinker functional groups, such that at least a portion of multifunctional monomer functional groups are covalently bound to crosslinker functional groups and at least a separate portion of multifunctional monomer functional groups are covalently bound to other multifunctional monomer functional groups.

In certain embodiments, the stiffness of the encapsulating matrix can be adjusted by altering the ratio of the multifunctional monomer and the crosslinker or by changing the identity of either species, including but not limited to increasing of decreasing the number of recpeating units or molecular weight of either species. In other embodiments, the stiffness of the encapsulating matrix can be adjusted by altering the water content of the composition. In other embodiments, the stiffness of the encapsulating matrix can be adjusted by exposing the microspheres to photoexcitation, whereby the photoexcitation increases induces additional crosslinking in the encapsulating matrix. In yet other embodiments, the photoexcitation induces crosslinking of at least a portion of multifunctional monomer functional groups with other multifunctional monomer functional groups. In yet other embodiments, the stiffness of the encapsulating matrix can be adjusted by exposing the encapsulating matrix to photoexcitation, whereby the photoexcitation degrades at least a portion of the degradable crosslinker in the encapsulating matrix. In yet other embodiments, the photoexcitation can be localized photoexcitation, allowing for spatiotemporal control of the stiffness of the encapsulating matrix.

In certain embodiments, the stiffness of the encapsulating matrix material is adjusted using a dual stage curing process.

In certain embodiments, the polymer microspheres and/or the encapsulating matrix further comprise at least one crosslinking initiator. In other embodiments, the at least one crosslinking initiator is a photoinitiator. In other embodiments, the photoinitiator is one or more compounds selected from the group consisting of Eosin-Y, 1-[4-(2-Hydroxyethoxy)-phenyl]-2-hydroxy-2-methyl-1-propane-1-one (I2959), acetophenone, anisoin, anthraquinone, anthraquinone-2-sulfonic acid, (benzene) tricarbonylchromium, benzyl, benzoin, benzoin ethyl ether, benzoin isobutyl ether, benzoin methyl ether, benzophenone, benzophenone/1-hydroxycyclohexyl phenyl ketone, 3,3′,4,4′-benzophenonetetracarboxylic dianhydride, 4-benzoylbiphenyl, 2-benzyl-2-(dimethylamino)-4′-morpholinobutyrophenone, 4,4′-bis(diethylamino)benzophenone, 4,4′-bis(dimethylamino)benzophenone, camphorquinone, 2-chlorothioxanthen-9-one, dibenzosuberenone, 2,2-diethoxyacetophenone, 4,4′-dihydroxybenzophenone, 2,2-Dimethoxy-2-phenylacetophenone, 4-(Dimethylamino) benzophenone, 4,4′-Dimethylbenzil, 2,5-Dimethylbenzophenone, 3,4-Dimethylbenzophenone, Diphenyl(2,4,6-trimethylbenzoyl)phosphine oxide/2-Hydroxy-2-methylpropiophenone, 50/50 blend 4′-Ethoxyacetophenone, 2-Ethylanthraquinone, Ferrocene, 3′-Hydroxyacetophenone, 4′-Hydroxyacetophenone, 3-Hydroxybenzophenone, 4-Hydroxybenzophenone, 1-Hydroxycyclohexyl phenyl ketone, 2-Hydroxy-2-methylpropiophenone, 2-Methylbenzophenone, 98% 3-Methylbenzophenone, Methybenzoylformate, 2-Methyl-4′-(methylthio)-2-morpholinopropio-phenone, 1-[4-(2-hydroxyethoxy)-phenyl]-2-hydroxy-2-methyl-1-propanone, bis(2,4,6-trimethylbenzoyl)-phenylphosphineoxide and lithium phenyl-2,4,6-trimethylbenzolphosphinate (LAP). In other embodiments, the at least one crosslinking initiator is a thermal or redox initiator. In yet other embodiments, the thermal or redox initiator is one or more compounds selected from the group consisting of 4,4′-Azobis(4-cyanovaleric acid), 4,4′-Azobis(4-cyanovaleric acid), 1,1′-Azobis(cyclohexanecarbonitrile), Azobisisobutyronitrile, 2,2′-Azobis(2-methylpropionamidine) dihydrochloride, 2,2′-Azobis(2-methylpropionitrile), 2,2′-Azobis(2-methylpropionitrile), ammonium persulfate, hydroxymethanesulfinic acid, potassium persulfate sodium persulfate, tert-butyl hydroperoxide, tert-butyl peracetate, cumene hydroperoxide, 2,5-di(tert-butylperoxy)-2,5-dimethyl-3-hexyne, dicumyl peroxide, 2,5-bis(tert-butylperoxy)-2,5-dimethylhexane, 2,4-pentanedioneperoxide, 1,1-bis(tert-butylperoxy)-3,3,5-trimethylcyclohexane, benzoyl peroxide, 2-butanone, tert-butyl peroxide, lauroyl peroxide, tert-butyl peroxybenzoate, and tert-butylperoxy 2-ethylhexyl carbonate.

In another aspect, the invention provides structures comprising the encapsulating matrix material. In certain embodiments, the structure comprises encapsulating matrix and at least one cell, as outlined elsewhere herein. In certain embodiments, the structure is shaped in an alveoli-like structure. In other embodiments, the structure is formed by forming the matrix encapsulated polymer microspheres as described elsewhere herein and then degrading the degradable crosslinkers, thereby degrading the polymer microspheres but leaving intact the encapsulating matrix.

In certain embodiments, the polymeric alveoli-like structures and hydrogels of the invention can be used to culture cells in environments that mimic natural pulmonary environments. In other embodiments, the polymeric alveoli-like structures and hydrogels of the invention can simulate the properties of healthy lung tissue (˜1-5 kPa) and fibrotic lung tissue (>20 kPa).

### Cell Culturing Methods

In one aspect, the invention provides methods of growing, expanding and culturing cells in the microspheres of the invention. In certain embodiments, the methods can be used to develop in vitro lung models.

In certain embodiments, the method comprises seeding cells in a uniformly dispersed polymer microsphere composition of the invention, incubating the cells in the uniformly dispersed polymer microsphere composition for a period of time, aggregating portions of the uniformly dispersed polymer microsphere composition to form alveoli-like clusters, encapsulating the alveoli-like clusters in an encapsulating matrix material and incubating the cells in the encapsulating matrix.

In certain embodiments, the cells are selected from the group consisting of basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells airway or bronchial epithelial cells and cell lines comprising A549, MLE-12 and/or 3T3 fibroblasts.

In certain embodiments, the aggregation of portions of the uniformly disperse polymer microsphere composition comprises magnetically levitating the microspheres and cells to form aggregates.

In certain embodiments, incubating the cells in the encapsulating matrix degrades the degradable crosslinkers thereby degrading the polymer microspheres but leaving intact the encapsulating matrix. In other embodiments, the incubating cells secrete enzymes that degrade the degradable crosslinkers. In other embodiments, UV light is applied to the encapsulated microspheres, degrading photodegradable crosslinkers, thereby degrading the polymer microspheres while leaving the encapsulating matrix intact.

In certain embodiments, the methods are suitable for growing cells in in vitro environments that closely resemble natural in vivo lung tissue. In other embodiments, the stiffness of the microspheres and/or the alveoli-like structures are altered to mimic softer, healthy tissue (about 1 kPa to about 5 kPa) or diseased lung suffering from pulmonary fibrosis (about 20 kPa to about 100 kPa).

In certain embodiments, the method allows for the development of the cells to be observed as they proliferate and grow. In other embodiments, the method allows for cell differentiation to be tracked and observed.

In certain embodiments, the method further comprises testing the encapsulated cells for the presence of at least one biological markers. In other embodiments, the at least one biological marker includes expressed RNA, mRNA, genes, soluble proteins, membrane-bound proteins, ECM proteins, ECM-bound proteins, cytokines, growth factors, enzymes, hormones, signaling ions, DNA content, metabolic byproducts, apoptosis markers, cell senescence markers, cell motility markers epigenetic changes, and contents of extracellular vesicles released by the cells.

In yet other embodiments, the encapsulated cells are tested for the expression of at least one marker selected from the group consisting of pro-fibrotic genes such as Acta2 (α-SMA), Agt, Ccl11 (eotaxin), Ccl12 (MCP-5, Scya12), Ccl3 (Mip-1a), Ctgf, Grem1, Il13, Il13ra2, Il4, Il5, and Snai1 (Snai1); anti-fibrotic genes such as Bmp7, Hgf, Ifng, 1110, and Il3ra2; extracellular matrix (ECM) structural constituents such as Col1a2, Col3a1; extracellular matrix (ECM) remodeling enzymes such as Lox, Mmp1a, Mmp13, Mmp14, Mmp2, Mmp3, Mmp8, Mmp9, Plat (tPA), Plau (uPA), Plg, Serpina1a, Serpine1 (PAI-1), Serpinh1 (Hsp47), Timp1, Timp2, Timp3, and Timp4; cell adhesion molecules such as Itga1, Itga2, Itga3, Itgav, Itgb1, Itgb3, Itgb5, Itgb6, and Itgb8; inflammatory cytokines & chemokines such as Ccl11 (eotaxin), Ccl12 (MCP-5, Scya12), Ccl3 (Mip-1a), Ccr2, Cxcr4, Ifng, 110, Il13, Il3ra2, Il1a, Il1b, 114, 115, Ilk, and Tnf; growth factors such as Agt, Ctgf, Edn, Egf, Hgf, Pdgfa, Pdgfb, and Vegfa; TGFβ signal pathway ligands such as Bmp7, Cavl, Dcn, Eng (Evi-1), Grem1, Inhbe, Ltbp1, Smad2 (Madh2), Smad3(Madh3), Smad4 (Madh4), Smad6, Smad7, Tgfb1, Tgfb2, Tgfb3, Tgfbr1 (ALK5), Tgfbr2, Tgif1, Thbs1 (TSP-1), and Thbs2; transcription factors such as Cebpb, Jun, Myc, Nfkb1, Sp1, Stat1, and Stat6; epithelial-to-mesenchymal transition (EMT) markers such as Akt1, Bmp7, Col1a2, Col3a1, Itgav, Itgb1, Mmp2, Mmp3, Mmp9, Serpine1 (PAI-1), Smad2 (Madh2), Snai1 (Snai1), Tgfb1, Tgfb2, Tgfb3, and Timp1; and other fibrosis genes, including Bcl2, and Fasl (Tnfsf6).

### Treatment Methods

The invention further provides a method of treating a disease or disorder in a subject in need thereof, the method comprising administering a polymer microsphere composition of the invention to the subject.

In one aspect, the invention provides a method of delivering cells to a subject through administration of the cell laden microspheres. In certain embodiments, the polymer microspheres comprise at least one cell, such as, but not limited to basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells airway or bronchial epithelial cells and cell lines comprising A549, MLE-12 and/or 3T3 fibroblasts.

In another aspect, the invention provides a method of delivering a pharmaceutical agent, growth factor, cytokine, or any other biochemical agent, to a subject. In certain embodiments, the polymer microspheres comprise at least one pharmaceutical agent, growth factor, cytokine, or any other biochemical agent for treatment of a disease.

In certain embodiments, the polymer microspheres composition is formulated as part of a pharmaceutical composition. In other embodiments, the pharmaceutical composition comprises at least one pharmaceutically acceptable carrier.

### Combination and Concurrent Therapies

In one embodiment, the compositions of the invention are useful in the methods of present invention when used concurrently with at least one additional compound useful for preventing and/or treating diseases and/or disorders contemplated herein.

In one embodiment, the compositions of the invention are useful in the methods of present invention in combination with at least one additional compound useful for preventing and/or treating diseases and/or disorders contemplated herein.

These additional compounds may comprise compounds of the present invention or other compounds, such as commercially available compounds, known to treat, prevent, or reduce the symptoms of diseases and/or disorders contemplated herein. In certain embodiments, the combination of at least one compound of the invention or a salt thereof, and at least one additional compound useful for preventing and/or treating diseases and/or disorders contemplated herein, has additive, complementary or synergistic effects in the prevention and/or treatment of diseases and/or disorders contemplated herein.

As used herein, combination of two or more compounds may refer to a composition wherein the individual compounds are physically mixed or wherein the individual compounds are physically separated. A combination therapy encompasses administering the components separately to produce the desired additive, complementary or synergistic effects.

In one embodiment, the compound and the agent are physically mixed in the composition. In another embodiment, the compound and the agent are physically separated in the composition.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114:313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The therapeutically effective amount or dose of a compound of the present invention depends on the age, sex and weight of the patient, the current medical condition of the patient and the progression of a disease or disorder contemplated in the invention.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the compositions of the invention are formulated using at least one pharmaceutically acceptable excipients or carriers. In one embodiment, the pharmaceutical compositions of the invention comprise a therapeutically effective amount of a compound of the invention and a pharmaceutically acceptable carrier.

The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical administration. In certain embodiments, routes of administration of any of the compositions of the invention include nasal, inhalational, intratracheal, intrapulmonary, and intrabronchial.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present invention are not limited to the particular formulations and compositions that are described herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Synthesis of Four-Armed PEG-Norbornene

Norbornene-functionalized PEG was prepared by the addition of norbornene acid via the symmetric anhydride N,N′-dicyclohexylcarbodiimid (DCC; Sigma) coupling. The 4-arm PEG, MW 20000 (JenKemUSA, Allen, Tex.), was dissolved in dichloromethane (DCM) with 5× (with respect to hydroxyls) pyridine and 0.5×4-(dimethylamino)pyridine (DMAP; Sigma). In a separate reaction vessel, DCC 5× with respect to PEG hydroxyls, was reacted at room temperature with 10×5-norbornene-2-carboxylic acid (Sigma). A few seconds after addition of the acid, a white byproduct precipitate formed (dicycolhexylurea), indicating the formation of dinorbornene carboxylic acid anhydride. The anhydride was allowed to stir for 30 min, following which the 4-arm PEG, pyridine, and DMAP solution were added. The reaction was stirred overnight, after which the mixture was filtered. The filtrate was washed with 5% sodium bicarbonate solution and the product was precipitated in ice-cold diethyl ether.

### Synthesis of Eight-Armed PEG-Norbornene

The reaction was carried out under anhydrous conditions in the organic solvent dichloromethane (DCM), where a PEG solution was added drop-wise to a stirred solution of N,N′-dicyclohexylcarbodiimide (DCC) and norbornene acid, and allowed to react overnight at room temperature. The norbornene functionalized PEG in this solution was then precipitated in ice-cold ethyl ether, filtered, and re-dissolved in chloroform. This chloroform PEG solution was then washed with a glycine buffer and brine before being precipitated in ice-cold ethyl ether and filtered again. The filtered PEG was then placed in a vacuum chamber to remove excess ether.

### Synthesis of Lithium Phenyl-2,4,6-Trimethylbenzoylphosphinate (LAP)

LAP was synthesized following existing protocol. Briefly, an equimolar amount of 2,4,6-trimethylbenzoylphosphonite was added to dimethyl phenylphosphonite and stirred for 18 hours. In a separate flask, a 4-fold molar excess of lithium bromide with respect to dimethyl phenylphosphonite was dissolved in 100 mL of 2-butanone. This mixture was stirred until the solute fully dissolved, then added to the previous reaction mixture. The reaction was then heated to 50° C. and a precipitate was observed after about 10 minutes. The reaction was removed from heat and allow to cool to room temperature for one hour. The product was filtered using a Buchner funnel, then washed and refiltered with 2-butanone three times. The product was collected in a 50 mL Falcon tube and dried overnight in a dessicator.

Synthesis of Alpha-Methacrylate (aMA) Functionalized Polyethylene Glycol

PEG was dissolved in anhydrous dichloromethane, and NaH (3.0 eq. with respect to hydroxyl functionality equivalents) was added to the solution. Ethyl 2-(bromomethyl) acrylate (2.0 eq. with respect to hydroxyl functionality equivalents) was then added. The reaction was carried out at room temperature overnight. The mixture was then neutralized with acetic acid and filtered by vacuum filtration. The solvent was removed by rotary evaporation. After re-dissolving the product residue in tetrahydrofuran, the solution was precipitated with cold diethyl ether and vacuum dried to give the final product.

General Procedures for Fabrication of Alpha-Methacrylate (aMA) Functionalized Hydrolysis-Resistant, Spatiotemporally Addressable Hydrogels

Prepare Stock Solutions:

Dilute alpha-methacrylate (aMA) functionalized polyethylene glycol (PEG) monomer in phosphate buffered solution (PBS) to 20 wt %. Dilute multifunctional thiol crosslinker to appropriate concentration in PBS. For MMP degradable crosslinkers, dilute to 2 wt %; for dithiothreitol (DTT) dilute to 5 wt %; for linear PEG dithiols, dilute to 7.5 wt %. Dilute triethanolamine (TEtA) to 50 wt % in PBS. Dilute selected photoinitiator (e.g. lithium phenyl-2,4,6-trimethylbenzolphosphinate (LAP), Eosin-Y, 1-[4-(2-Hydroxyethoxy)-phenyl]-2-hydroxy-2-methyl-1-propane-1-one (I2959)) to 2.5 wt % in PBS.

Hydrogel Preparation Procedures:
a. Prepare gaskets to suitable size and volume for relevant application (50 μl, 7 mm diameter for rheology).
b. Combine alpha-methacrylate PEG monomer stock solution, thiol crosslinker stock solution, photoinitiator stock solution (final concentration 0.05 wt %), and residual PBS volume to achieve appropriate macromer wt % (5-10 wt %) and stoichiometry (thiol:aMA=3:8 through 5:8)
c. Add TEtA stock solution to concentration appropriate for gelation kinetics (0.05 M for DTT and PEG dithiol crosslinkers, 0.1-1 M for MMP-degradable crosslinkers)
d. Vortex combined solution and aliquot to gaskets.
e. Observe gelation (3-5 minutes, dependent on crosslinker choice and TEtA concentration).
f. Swell resulting hydrogel overnight in PBS containing equal concentration of photoinitiator (2.5 wt %) to mitigate diffusion of photoinitiator out of hydrogel.
g. Irradiate hydrogel at 10 mW/cm² for 5 minutes at wavelength appropriate for photoinitiator used (365 nm for LAP or 12959, 517 nm for Eosin Y)
h. Quantify elevated modulus in resulting hydrogel.

Briefly, the gaskets are used as molds to make hydrogel discs. The gaskets are pressed onto glass slides and the appropriate volume of hydrogel solution for the specific “mold” being used is then pipetted using a micropipette into the mold. Gelation occurs as base-catalyzed Michael addition progresses. In one embodiment, the gaskets are 1 mm thick polysiloxane sheets of 50A durometer. They are cut to the dimensions of a glass slide and then 7 mm disks are punched out of the gasket. The resulting gasket is a rectangle of the same width and length of a glass slide with a thickness of 1 mm, with −15 7 mm holes distributed throughout its area. The hydrogel solution is aliquoted into these holes.

Protocols for the Differentiation of Induced-Pluripotent Stem Cells (iPSC) into Mature Pulmonary Epithelial Cells

Induced-pluripotent stem cells (iPSC) were differentiated from a commercially available fibroblast line into NKX2-1⁺ cells using a modified protocol based on the protocols reported in Jacob, et al. Cell Stem Cell 2017, 21, 472.

First, iPSCs are differentiated into definitive endoderm using STEMdiff Definitive Endoderm Kit (Stem Cell Technologies). Then anterior foregut-like endoderm (AFE) are generated on 2D hydrogel by the addition of CHIR, BMP4, KGF, FGF10 and retinoic acid (RA). To enrich for NKX2-1⁺ cells, CD47^{high}/CD26^{low} cells are sorted for on day 15 of differentiation. The CD47^{high}/CD26^{low} cell population has been shown to be highly enriched for NKX2-1⁺ lung progenitors capable of maturing into SFTPC⁺ ATII cells. Following sorting, cells are transferred to 3D hydrogel cultures and maintained in media containing CHIR, FGF10 and KGF for 7 days followed by the addition of dexamethasone, cAMP, and IBMX up to 35 days to form organoid structures. Lung epithelial cell lineage are confirmed by quantifying expression of genes highly expressed in pulmonary epithelial cells: ATII (SFTPC, SFTPC, LAMP3, ABCA3) and ATI (AQP5, PDPN) specific markers by qRT-PCR and immunohistochemistry. The formation of lamellar bodies within alveolospheres developed in 3D Matrigel cultures are also assessed by fixing, sectioning and immunogold labeling for SFTPB and SFTPC for electron microscopy.

### Example 1: Synthesis of Lung Mimic Biomaterials

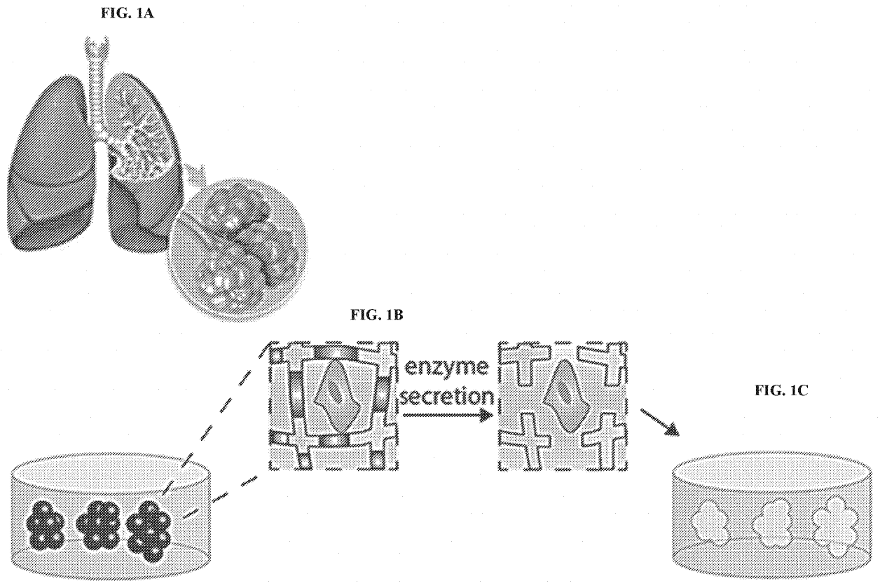

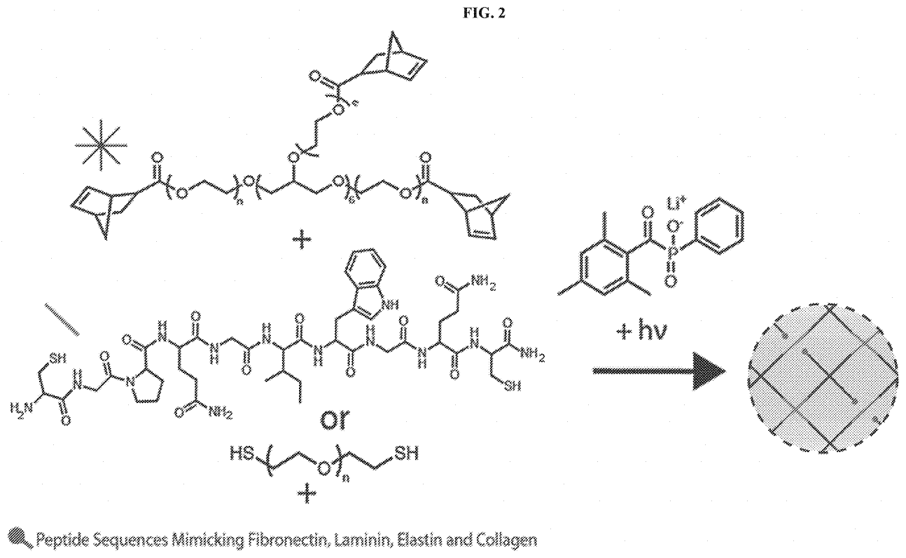

Hydrogel systems and microfabrication techniques are developed to allow for the development of 3D models of healthy and fibrotic lung tissue (FIGS. 1A-1C). Hydrogel precursor materials such as poly(ethylene glycol)-norbornene (PEG-NB) are synthesized and reacted with a degradable crosslinker, such as CGPQGIWGQGC peptide crosslinker and a peptide sequence mimicking the cell-adhesive protein fibronectin as shown in FIG. 2 in order to form a hydrogel microsphere template. Molecular weights and concentrations of hydrogel precursors and peptide crosslinker sequences are varied to tune biodegradation rates and enable primary lung cell engraftment within the 3D model.

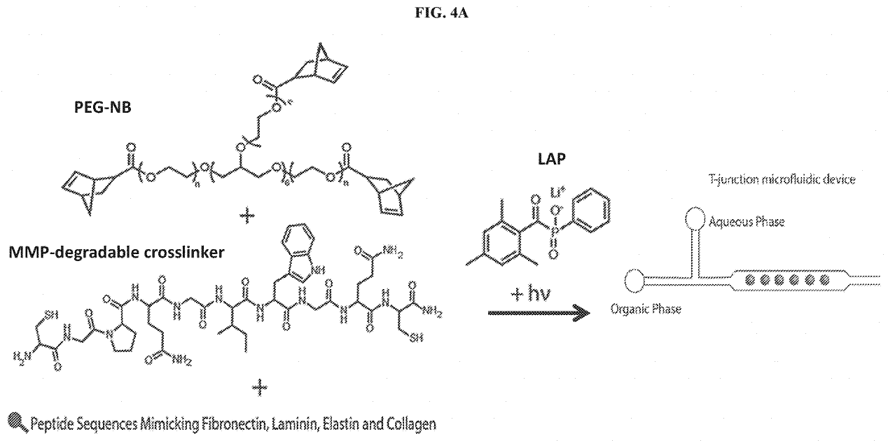

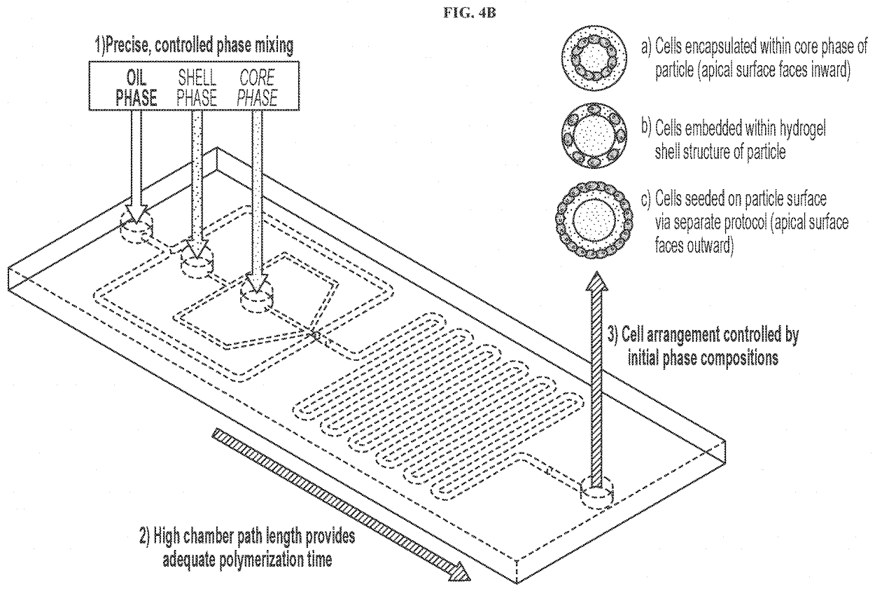

Biodegradable PEG-NB-based hydrogel microspheres can be synthesized using emulsion polymerization techniques. However, these methods do not produce highly uniform microspheres necessary to mimic alveolar structure. Alternatively, microfluidic devices are used as they have been shown to provide adjustable, consistent and high-throughput methods for fabricating monodisperse microspheres (FIG. 4A). To overcome irregularities in emulsion polymerization, t-junction droplet breakup microfluidic devices with input channels of size 50, 100 and 200 m are designed, 3D-printed (Ember, Autodesk; SM-412 Flexible Elastomer, Colorado Photopolymer Solutions) and tested to evaluate the influence of channel size on microsphere diameter, size distribution and degradation rate. Briefly, an aqueous phase (PEG-NB, MMP-degradable crosslinker, peptide mimics and a photoinitiator dissolved in phosphate buffered saline (PBS)) and an organic phase (Tween 20 and Span 80 in hexane) are pumped into microfluidic devices (˜1 psi) (FIGS. 4A-4B). Microspheres formed in the channels are collected in a bath with the same composition as the organic phase and exposed to UV light (365 nm, 10 mW/cm² for 2 minutes; Omnicure S2000, Lumen Dynamics) to photopolymerize.

Referring now to FIG. 4B, in an exemplary embodiment, monodisperse biodegradable PEG-NB-based hydrogel core-shell microparticles can be fabricated through the use of microfluidic devices. Microparticles made through the use of a microfluidics device can possess additional advantages over microspheres made through other means, including for example the ability to control the spatial orientation of basal and apical cell surfaces in relation to the lung mimic structure. The exemplary microfluidic device is designed with experimentally determined channel dimensions and provides precise control of core-shell microparticle size. An ageous core phase of culture media or PBS, an ageous shell phase of PEG-NB, a biodegradable crosslinker, peptides, and a photoinitiator (LAP), and a hydrophobic oil phase are mixed in precise flow quantities at the flow-focusing junction. Viscosity is modified to limit mixing of aqueous phases. The mixed phases then proceed through the remainder of the channel length under UV irradiation (365 nm, 10 mW/cm² for 2 minutes; Omnicure S2000, Lumen Dynamics). Particle size is determined by design of the microfluidic device. Conversely, shell thickness can be modified through modification of the shell phase flow rate. Spatial orientation of cells is determined by initial conditions. When cells are incorporated in the core phase, cells adhere to the interior wall of the shell, resulting in the apical surface oriented toward the center of the microparticle. If cells are incorporated in the shell phase, cells are embedded in the hydrogel shell matrix of the particle. Cells can also be seeded on microparticles post-fabrication, resulting in the apical surface oriented away from the center of the particle.

Microsphere or core-shell microparticle sizes and degradation rates are evaluated over 14 days by analyzing images of samples that have been fluorescently tagged with AlexaFluor 488 C5 maleimide through covalent bonding with free thiols in the polymer system. Day 0 measurements represent initial microsphere or core-shell microparticle size. Then microspheres or core-shell microparticles are stored in a solution of collagenase (Type II, 5 U/ml) to stimulate MMP degradation or PBS as a control at 37° C. Samples are collected and imaged using fluorescent microscopy every two days. Image J software is used to measure dimensions of at least 300 microspheres or core-shell microparticles from three replicates of each condition at each time point. Results are analyzed to improve biodegradable microsphere or core-shell microparticle formulation and microfabrication until 200-μm microspheres that degrade completely over the 14-day time period are produced consistently. Altering the ratio of polymer precursors, changing the sequence of the MMP-degradable crosslinker and/or adjusting the width of the channels in the microfluidic devices can achieve this goal.

### Example 2:3D Cell Culture Microenvironments

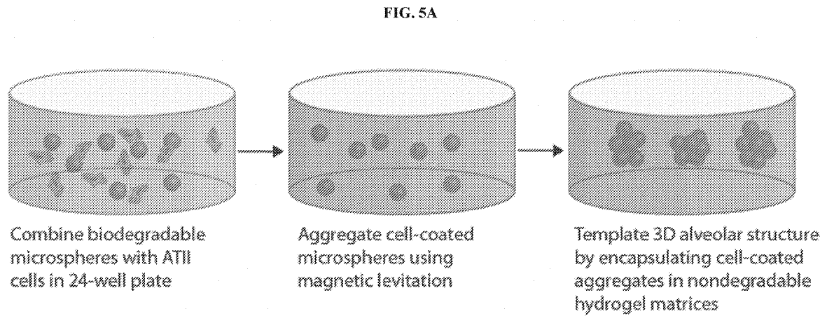

Primary murine ATII cells are isolated to elucidate the impact of microenvironmental stiffness on ATII phenotype and signaling. Cells are dissociated from lung tissue and sorted by negative selection through incubation with antibodies (CD16/32: B-cells, monocyte/macrophages, NK cells, and neutrophils. CD45: hematopoietic cells. CD90: T-cells, TER119: erythroid cells, fibroblasts) and adherence to isolate fibroblasts. Purity and viability of ATII cell preparations using these techniques are consistently greater than 90 and 95%, respectively with a yield of 2-3×106 cells per animal. To produce 3D cell culture platforms, microsphere templates, containing peptide sequences mimicking fibronectin, designed in Example 1 are seeded with primary ATII cells, by exposing 500,000 cells/ml to microspheres suspended in sterile cell culture media in an ultra-low adhesion 24-well plate (FIG. 5A). The plates are placed on an orbital shaker at 45 rpm and incubated at 37° C. with 5% CO₂ overnight. Following incubation with cells for 72 h, microspheres are aggregated into structures that mimic alveolar clustering in vivo using magnetic levitation. Cells are magnetized by exposure to nanoparticle assemblies of gold, iron oxide and poly-L-lysine, which bind nonspecifically to cell membranes (NanoShuttle, Greiner Bio-One) and cell-laden microsphere templates are aggregated using specialized magnetic cell culture plates (Bio-Assembler Kit, Greiner Bio-One).

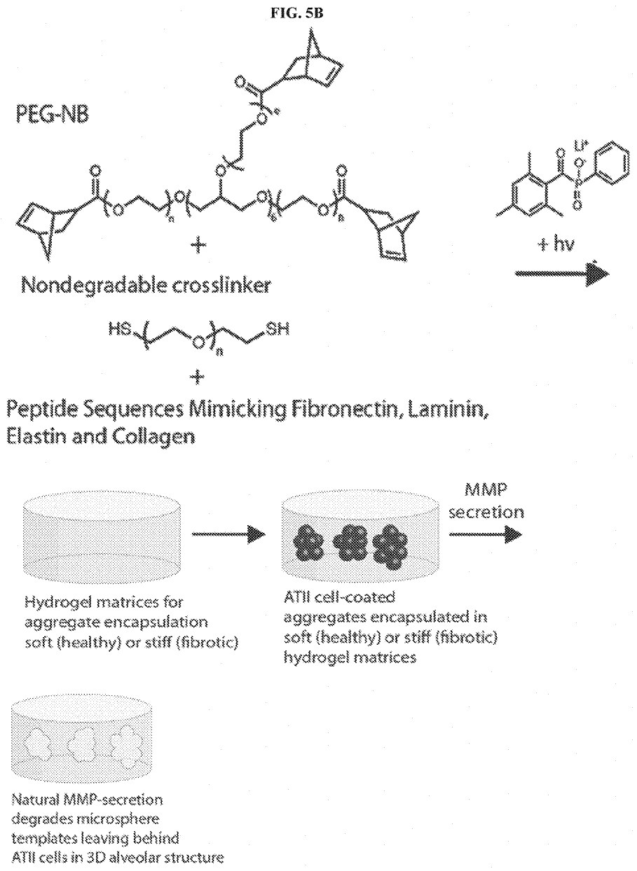

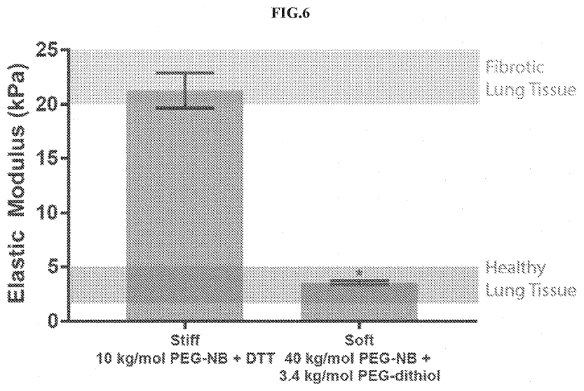

Lung tissue ranges in stiffness from 5 kPa (healthy) to 20 kPa (fibrotic). Preliminary data show that the molecular weight of the PEG-NB macromer can be adjusted to achieve stiffness values within this range (FIG. 6). Encapsulating hydrogel matrices are synthesized using 8-arm, 10 kg/mol PEG-NB and a non-degradable PEG-dithiol crosslinker to reproduce the stiff microenvironment that has been reported for fibrotic lung tissue or 40 kg/mol for healthy tissue. Stiffness of the new materials is verified by rheology. Briefly, freestanding films of each hydrogel formulation (N=5) are cast between two siliconized glass slides to produce discs (height: 1 mm; diameter: 7 mm), which are swollen to equilibrium for bulk rheological measurements. The storage and loss moduli (i.e., G′ and G″) are quantified for 3 replicates from each batch of polymer on a parallel plate rheometer (DHR-3, TA Instruments) equipped with an 8-mm plate. Hydrogels are subjected to oscillatory shear at 1% strain through a dynamic angular frequency range to measure mechanical properties. Aggregated structures are then encapsulated within these material systems to evaluate the impact of varying local stiffness on induction of profibrotic ATII phenotype (FIG. 5A).

ATII cell viability, arrangement and polarization in 3D are monitored over a time period of up to 28 days in culture. A Live/Dead cell viability assay kit (ThermoFisher) is used to stain hydrogels at various time points (Day 3, 7, 14, 21, 28). Cell culture platforms (N=3, per condition, at each time point) are immediately imaged on a confocal microscope (Zeiss LSM 710), and image stacks are analyzed for red cell count (dead) and green cell count (live) with ImageJ (NIH). Likewise, cell culture platforms are stained to visualize E-cadherin, T1α, surfactant protein C and cell nuclei at the same time points (N=3, per condition, at each time point) and imaged on a confocal microscope to visualize epithelial cell arrangement and polarization and confirm replication of alveolar structures in 3D.

### Example 3: Fabrication of Biomaterials for Use in Generating Mature Pulmonary Epithelial Cells

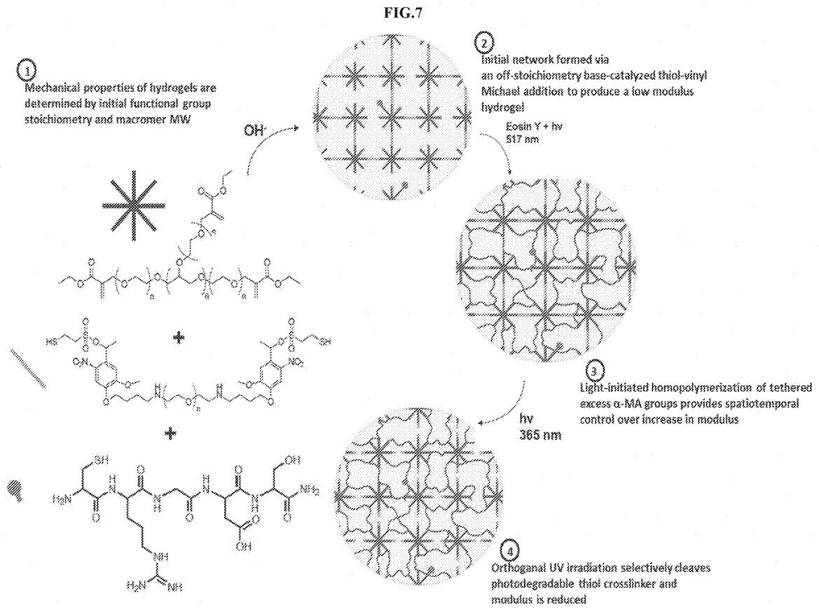

Poly(ethylene glycol) (PEG)-a-methacrylate macromers are reacted via Michael addition with a dithiol crosslinker, for example PEG dithiols, dithiothreitol or CGPQGIWGQGC peptide, and a peptide sequence that mimics the adhesion protein fibronectin (CGRGDS) to create an initially soft, cell adhesive hydrogel matrix (FIG. 7). The initial reaction is performed off-stoichiometry leaving excess methacrylate groups free for a secondary polymerization reaction. At a later time point, a photoinitiator can be swollen into the system and initiated with cytocompatible ultraviolet light (365 nm) to stiffen the hydrogel matrix with spatiotemporal control (FIGS. 7 and 12B).

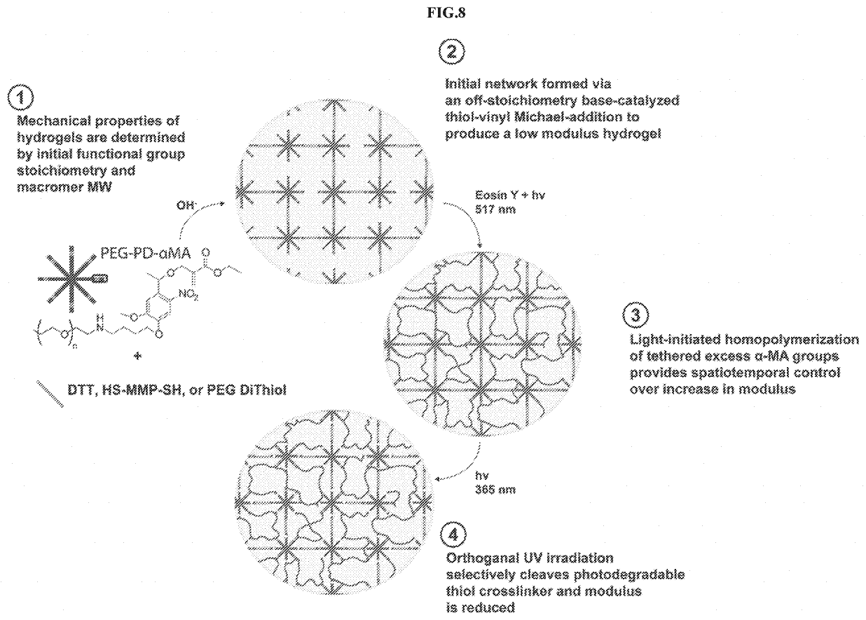

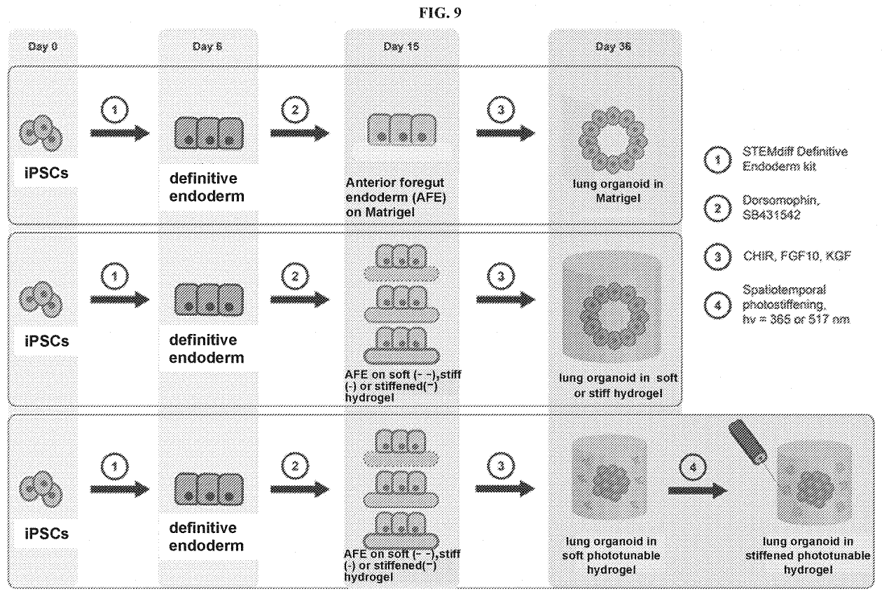

Both 2D and 3D cell culture platforms can be made from these materials to create soft, stiff and temporally stiffened microenvironments for incorporation into induced-pluripotent stem cells (iPSC) to lung epithelium differentiation and organoid formation protocols as outlined in FIG. 9. Stiffness of the new materials and comparisons to traditional Matrigel substrates are determined by rheology. Briefly, freestanding films of each hydrogel formulation are cast between two siliconized glass slides to produce discs. The discs are then swollen to equilibrium for bulk rheological measurements. The storage and loss moduli (i.e., G′ and G″) are quantified for at least 3 replicates from each condition on a parallel plate rheometer (DHR-3, TA Instruments).

The novel hydrogel biomaterials are then incorporated into iPSC differentiation protocols as described elsewhere herein and compared with Matrigel controls. Without intending to be limited to any particular theory, culturing iPSCs on soft hydrogel substrates is more likely to cause greater differentiation into ATII cells, while culturing on stiffer hydrogel substrates is more likely to cause greater differentiation into ATI cells. qRT-PCR is performed for markers of ATI and ATII cell differentiation. Lamellar bodies in organoids generated within the hydrogel materials of the invention are compared with those generated using a Matrigel control. SFTPB and SFTPC in sectioned alveolospheres are immunogold labeled and the expression of these factors is compared between organoids grown in Matrigel and organoids grown in the hydrogels of the invention.

### Example 4: Fibrosis Models Using Novel Hydrogel Biomaterials

### Initiation of Profibrotic Phenotype in Encapsulated Cells Through Local Microenvironment Stiffening

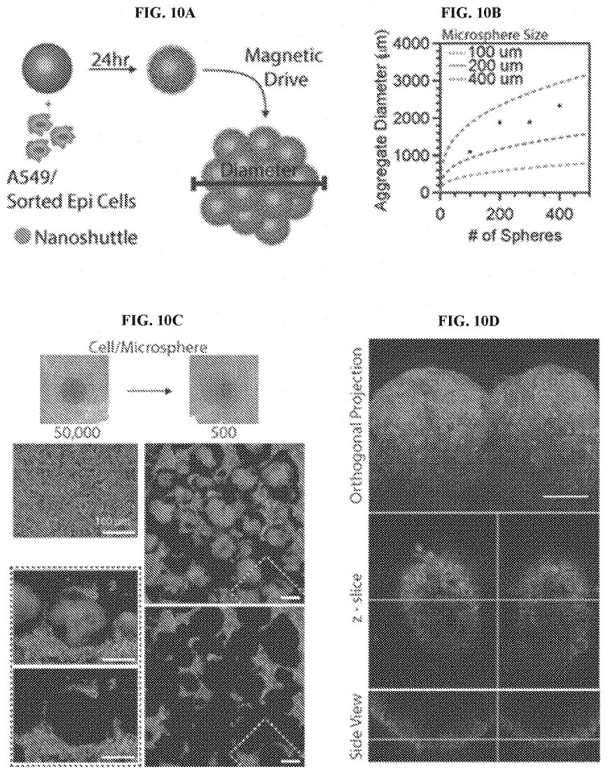

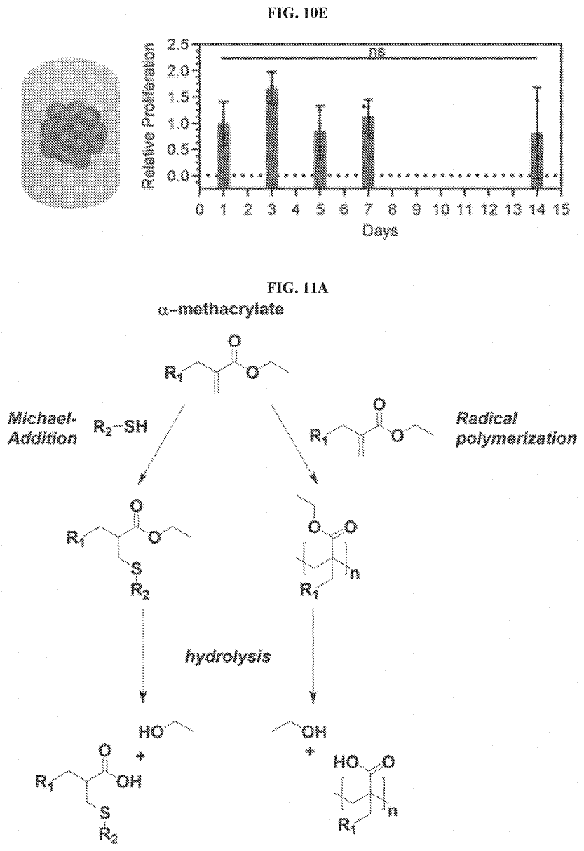

A cell-templating technique that mimics distal lung geometry in 3D (FIGS. 10A-10E) for improving organoid formation was developed. First, matrix metalloproteinase (MMP)-degradable, PEG microspheres are synthesized via emulsion polymerization and then seeded with pulmonary epithelial cells derived from iPSCs, as described elsewhere herein. Cell-microsphere complexes are aggregated by magnetic levitation to form alveoli-like structures and subsequently embedded within a dual-stage polymerization hydrogel of the invention with or without fibroblasts, derived from the same iPSC line in the encapsulating matrix. Once 3D cultures have been established (Day 20) in soft matrices, half of the matrices are stiffened in situ to simulate development of fibrosis.

At the completion of each experimental time point (21, 28 and 35 days) samples (n=6) are cryosectioned into thin slices for histology or processed for gene expression. Two assays are performed on histological sections: 1) a Ki67 immunoassay is used to detect proliferating cells in G1, S, G2 and M phases, and 2) sections of 3D cell culture platforms are stained and evaluated by image analysis for expression of elastin, collagen types I and V, α-smooth muscle actin and tenascin C, which have all been demonstrated to increase on the protein level during fibrotic pathology. Additionally, the Human Fibrosis RT² Profiler PCR Array (QIAGEN) is used to interrogate expression of 84 key genes involved in dysregulated tissue remodeling during fibrosis from each of these sample areas. The array contains assays for profibrotic genes (e.g., Acta2, CTGF, Snai1) as well as genes encoding for ECM remodeling enzymes (i.e. MMPs), TGF-β signaling molecules and inflammatory cytokines. Results from experimental conditions using novel hydrogel biomaterials are compared to organoids developed in 3D Matrigel controls with or without TGF-β treatment, a soluble factor commonly used to induce profibrotic cellular activation in vitro. Statistical analysis including one-way analysis of variance (ANOVA) and Tukey's post hoc tests for multiple comparisons or paired t-tests are performed as applicable on every data set and provide the foundation for an iterative design process, including controlled modification, systematic testing and iterative improvement, to optimize microenvironments to mimic the hallmarks of IPF pathobiology.

### Responsiveness of IPF Models to Standard of Treatment Therapeutics

The novel hydrogel biomaterials of the invention are exposed to currently available IPF therapeutics (e.g. Pirfenidone and Nintedanib) to demonstrate that the models can be used for high-throughput screening of therapeutics. Briefly, replicates of the model systems (n=6, for each condition and time point) are cultured until fibrotic phenotypes are achieved, dosed with therapeutics as recommended by the manufacturers and reassessed for fibrotic markers as outlined elsewhere herein. Statistical analysis of the results confirms the potential for these model systems to be used to recapitulate reduction in fibrosis measured in vivo upon treatment with these therapies. Reduction of fibrotic phenotype can suggest that it is feasible to use the bio-inspired 3D cell culture platforms of the invention as high-throughput screens for precision medicine.

### Example 5: Fabrication of Synthetic 3D Templates that can be Used to Pattern Primary Lung Cells within a Well-Defined Hydrogel Matrix that Mimics Healthy or Fibrotic ECM

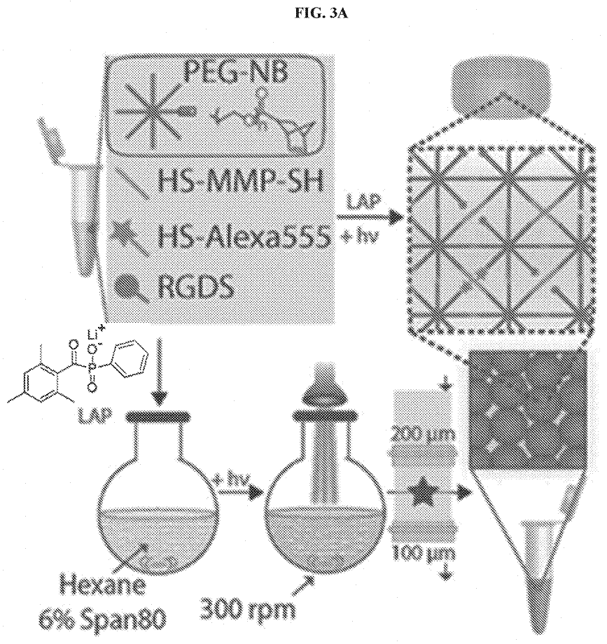

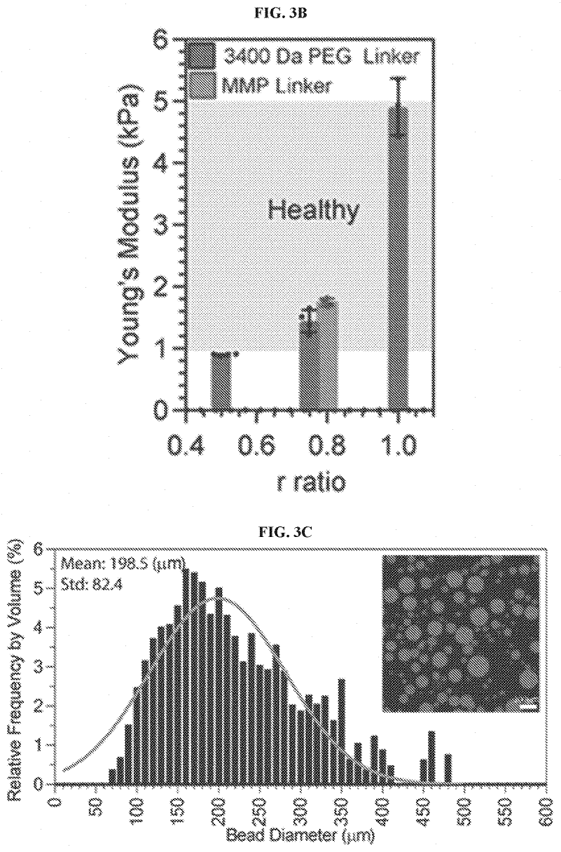

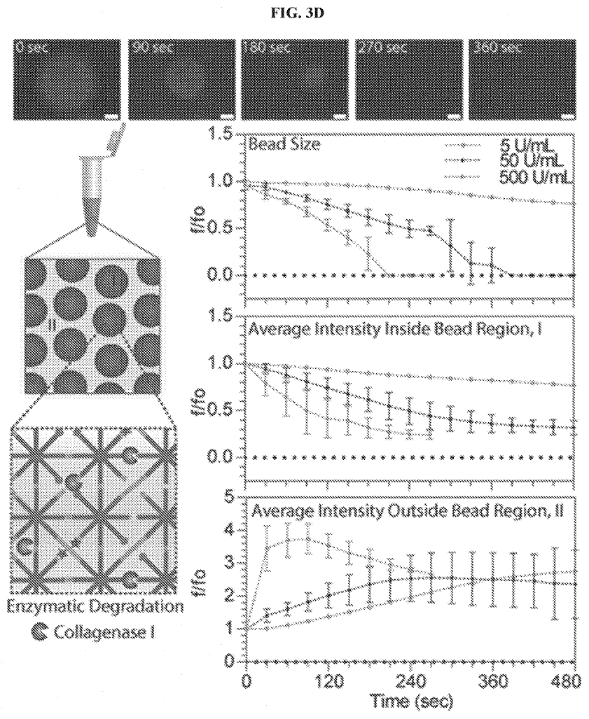

The natural structure of the alveolar space is mimicked by aggregating degradable hydrogel microspheres. Matrix metalloproteinase (MMP) degradable thiol-ene polyethylene glycol (PEG) hydrogel microspheres, synthesized via an inverse suspension polymerization method (FIG. 3A), are aggregated using magnetic nanoparticles and magnetic fields generated by a magnet to levitate the cell/microsphere solution. This synthetic template platform gives control over the material mechanical properties. The ratio of the reactants can be varied to achieve a range of Young's moduli (FIG. 3B) which allowed to tool the microsphere mechanicals into a range experienced by epithelial cells within health tissue in vivo.

Eight-armed PEG-Norbornene (NB) (40 kg/mol) was combined with MMP-degradable crosslinker peptide (CGGPQGIWGQGC) (GL Biochem, Boston, Mass.) in HEPES buffer at a final gel composition of 1.22 mM PEG-NB, 3.89 mM crosslinker, 1 mM RGD (CGRGDS), 1 mM YIGSR (CGYIGSR), and 2.2 mM LAP. The solution was pipetted into 6% Span 80/hexane solution at 6 ml to 10 ml hexane, vortexed, and exposed to 405 nm light at 20 mW/cm² for 10 min as depicted in FIG. 3A. The microspheres were filtered through 200 m and 100 m nylon filters to target an average microsphere size that mimicked alveolar structure (d˜200 μm). Hydrogel microspheres formed are 198.5±82.4 μm in diameter (FIG. 3C) and degradable by collagenase type I (FIG. 3D) providing the essential design criteria for an aveoli mimic.

To fully mimic the alveolar structure the hydrogel microspheres are coated with epithelial cells and aggregated using a magnetic field (FIG. 10A). A549 cells were initially evaluated to determine cell to microsphere concentrations to achieve monolayer cultures around microspheres and aggregate size dependence on number of microspheres. NanoShuttle (n3D Biociences, Inc.) (1 μl/1×10⁴ cells) is used to magnetize the cells which are then combined with microspheres at 500-50,000 cells/microsphere. After 24 h of culture with the microspheres, the magnetic drive was applied to aggregate the cells and microspheres.

The drive was removed for further culture of the aggregate before fixing and imaging or embedding and sectioning for analysis. The aggregate size is dependent on the number of microspheres and the microsphere size distribution (FIG. 10 B) and the cell/microsphere aggregates increased in cell density (darker aggregate) (FIG. 10 C) as the concentration of cells/microsphere increased, as expected. Monolayer coating of the microspheres is observed at the lower concentration range, 500 cells/microsphere (FIG. 10C, FIG. 10D). Once monolayer coated aggregates were achievable it was needed to confirm that this in vitro platform would be able to withstand long term culture. A 14 day viability study of encapsulated aggregates revealed the platforms ability to maintain viable cells over time (FIG. 10E).

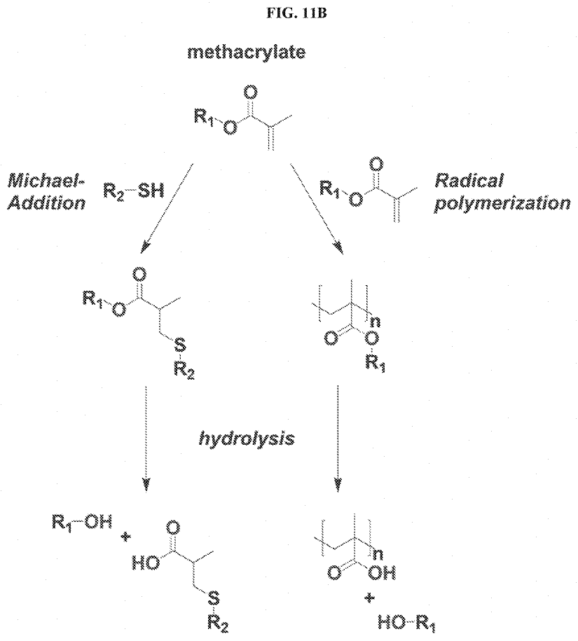

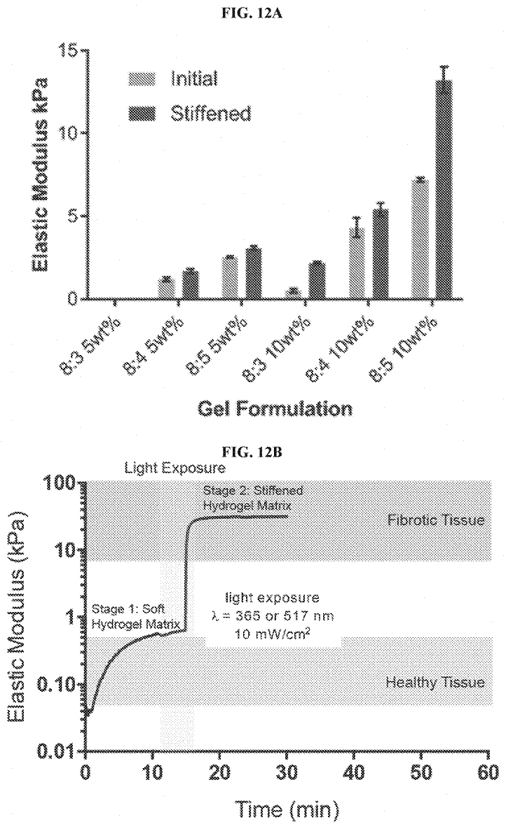

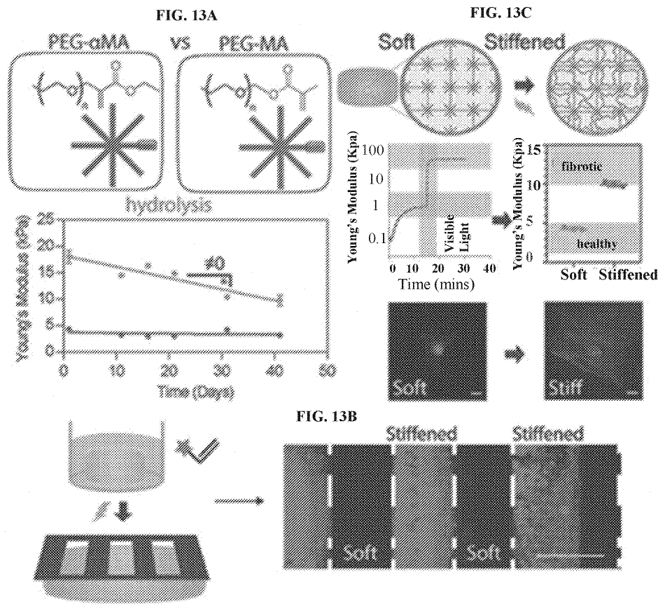

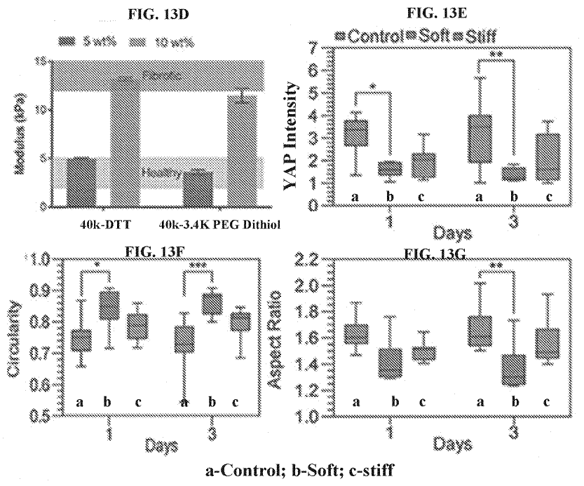

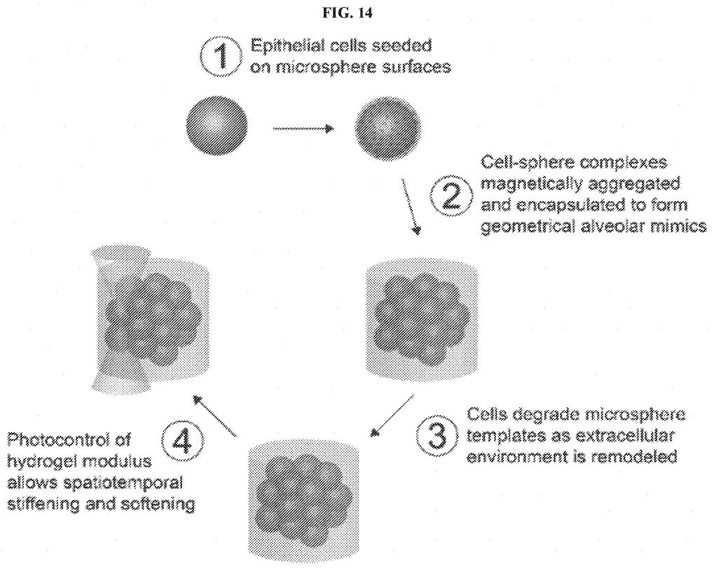

The encapsulating material was then developed considering that local tissue stiffness is strongly believed to be a driving force for the continuous alteration of cell phenotype and function. A new class of hydrolytically stable (FIG. 13A), phototunable poly(ethylene glycol) (PEG)-based hydrogel biomaterials that allows to control the mechanical properties of the local microenvironment on-demand around encapsulated cells using focused light (FIG. 13B) are developed. The PEG α-methacrylate (PEGαMA) macromer is synthesized by reacting PEG-hydroxyl (8-arm, 10 kg/mol; JenKem) with ethyl 2-(bromomethyl) acrylate in dichloromethane in the presence of sodium hydride. Hydrolytic stability is monitored by measuring the elastic modulus of the PEGαMA hydrogels stored in phosphate buffered saline at 37° C. compared to PEGMA controls and PEGαMA hydrogels resisted hydrolysis over 41 days compared to traditional PEGMA (FIG. 13A). In a dual-stage polymerization process 1) PEGαMA is reacted by Michael addition with dithiothreitol (DTT) at a ratio of 2 αMA:1 thiol to form a soft hydrogel and 2) a homopolymerization of free aMA moieties is initiated to stiffen the hydrogel (2.2 mM LAP, 10 mW/cm²). Dynamic mechanical properties were evaluated by parallel plate rheology (1% strain through a dynamic angular frequency range (0.1 to 100 rad s−1). This dual cure system allow to embed aggregates in initially soft (1-5 kPa) hydrogels that mimic healthy tissue and stiffen to emulate fibrotic progression (>10 kPa) (FIG. 13C).

Thus, the cell-degradable microspheres coated with primary lung epithelial cells are aggregated using magnetic levitation and embedded within the PEGαMA hydrogels. This novel biomaterial platform that can incorporate encapsulated fibroblast and can recapitulate time- and space-dependent changes in ECM mechanical properties finds application in understanding how the interfaces between fibrotic and healthy tissues influence disease progression and efficacy of drug delivery.

### Example 6: Evaluating Effects of Local Extracellular Stiffness on Primary ATII Cell Phenotype and Production of Profibrotic Mediators

Within the distal lung tissue fibroblast surround the epithelial cell lined alveolar structures. Hence it is important to understand how both cell types' phenotype is influenced by the local ECM mechanical properties. The effects of local ECM stiffness on the activation of fibroblasts and epithelial cells were evaluated. Immunofluorescent staining for α-smooth muscle actin (αSMA) of human lung fibroblasts on soft (1-5 kPa) vs stiff (>10 kPa) hydrogels mimicking healthy and fibrotic tissue, respectively, showed increased αSMA expression and organization on stiff substrates compared to soft hydrogels as expected (FIG. 13C). A549 cells (model of ATII cells) were initially evaluated on stiff (10 wt % 40k-DTT, FIG. 13D) and soft (5 wt % 40k-3.4k PEG Dithiol, FIG. 13D) hydrogels and the normalized YAP intensity (FIG. 13E), circularity (FIG. 13F), and aspect ratio (FIG. 13G) were all evaluated after 1 and 3 days in culture. When A549 cells were cultured on soft hydrogels as compared to the control (tissue culture plastic) there was a significant drop in the YAP intensity and the A549 cells took on a more rounded phenotype, as indicated by circularity and aspect ratio closer to 1. These results highlight the need to further understand how the local ECM mechanics fibroblast and epithelial cell phenotypes in the context of pulmonary fibrosis.

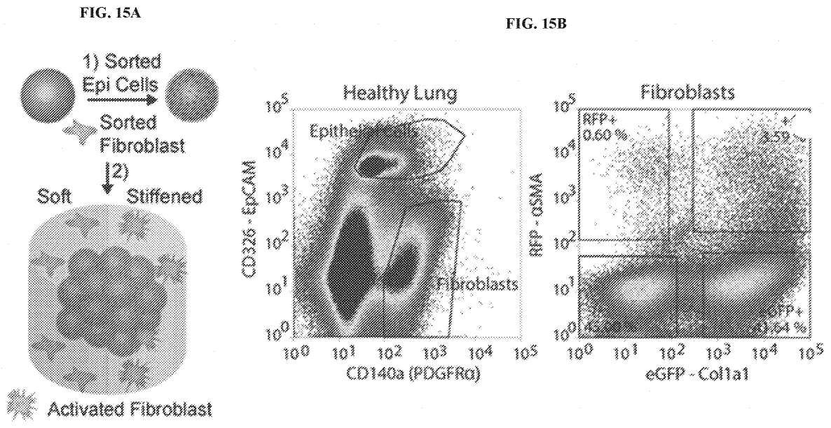

In this example, it was sought to evaluate primary lung fibroblast and ATII cells within the novel in vitro biomaterial platform, where time- and space-dependent changes in ECM mechanical properties enables not only the study of how the interfaces between fibrotic and healthy tissues influence disease progression but also enables the evaluation of efficacy of drug delivery (FIG. 15A). Using the bio-material platform of the invention, other cell types including the sorted primary lung epithelial cells, identified as lineage negative (CD31−, CD45− and PDGRFa−) and EpCAM+, and primary lung fibroblasts (PDGRFa+) simultaneously encapsulated within the embedding matrix can be further studied to evaluate the influence of epithelial-fibroblast crosstalk on initiation of fibrotic regions in vitro (FIG. 15B).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1.-72. (canceled)

73. A method of culturing cells in an in vitro tissue model, the method comprising:
incubating cells seeded in a uniformly dispersed polymer microsphere composition;
aggregating portions of the uniformly dispersed polymer microsphere composition to form alveoli-like clusters; and
encapsulating and incubating the alveoli-like clusters in an encapsulating matrix material;
wherein the polymer microspheres comprise: at least one multifunctional monomer; at least one peptide segment; and at least one degradable crosslinker;
wherein the encapsulating matrix material comprises: at least one multifunctional monomer; at least one crosslinker, wherein the at least one crosslinker is at least one non-degradable crosslinker, at least one degradable crosslinker, or at least one non-degradable crosslinker and at least one degradable crosslinker; and at least one peptide segment.

74. The method of claim 73, wherein the at least one multifunctional monomer is each independently selected from the group consisting of functionalized poly(ethylene glycol), poly(ethylene oxide), poly(vinyl alcohol), poly(vinyl acetate), poly(ethylene imine), polyacrylamide, poly(hydroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), acrylate-functionalized gelatin, methacrylate-functionalized gelatin, acrylate-functionalized hyaluronic acid, and methacrylate-functionalized hyaluronic acid;
wherein the at least one multifunctional monomer is each independently functionalized with at least one functional moiety selected from the group consisting of acrylate, methacrylate, norbornene, thiol, azide, alkene, alkyne, oxime, hydrozone, isocyanate, tetrazine, maleimide, vinyl sulphone, dibenzocyclooctyne and NHS-ester; and
wherein the at least one multifunctional monomer is each independently functionalized with at least two, at least three, at least four, or at least eight functional moieties.

75. The method of claim 73, wherein at least one multifunctional monomer is a compound of Formula (IA): 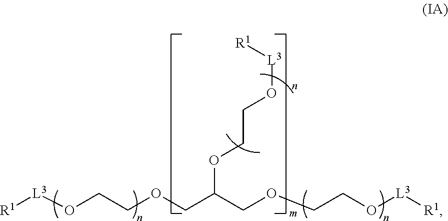
wherein:
each instance of L³ independently comprises a linkage selected from the group consisting of a bond, 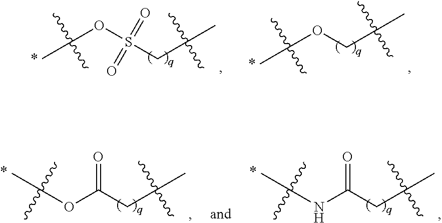
wherein the * side of the linkage is bound to the monomer and the opposite side is bound to R¹, and wherein q is an integer selected from 0 to 6;
each instance of R¹ independently comprises a functionality selected from the group consisting of acrylate, methacrylate, alpha-methacrylate, norbornene, thiol, azide, alkene, alkyne, oxime, hydrozone, isocyanate tetrazine, maleimide, vinyl sulphone, dibenzocyclooctyne, NHS-ester, 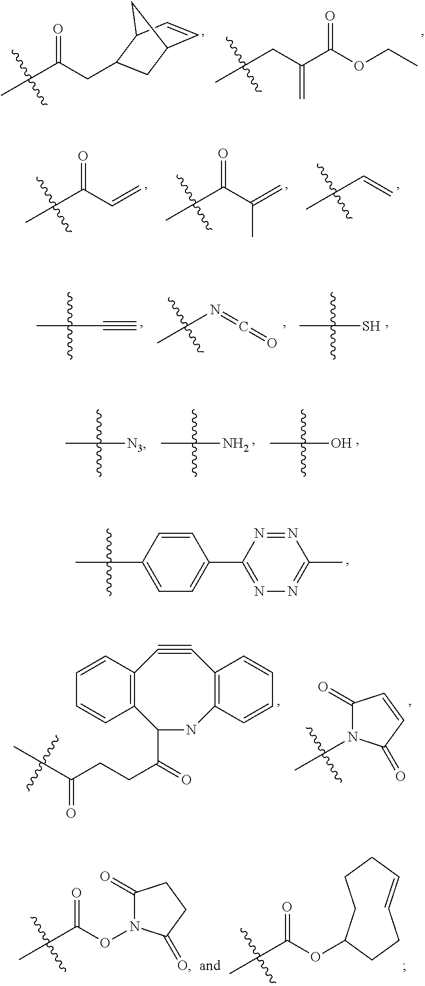
m is an integer from 0 to 10; and
n is an integer from 1 to 500.

76. The method of claim 73, wherein at least one peptide segment is a segment from at least one protein selected from the group consisting of matrisome protein and matrisome-associated protein.

77. The method of claim 73, wherein the cells are selected from the group consisting of basal stem cells, distal alveolar stem cells, induced pluripotent stem cells, fibroblasts, type I alveolar epithelial cells, type II alveolar epithelial cells, endothelial cells, endothelial progenitor cells, mesenchymal stem cells, airway or bronchial epithelial cells and cell lines comprising A549, MLE-12 and/or 3T3 fibroblasts.

78. The method of claim 73, wherein the at least one non-degradable crosslinker is selected from the group consisting of functionalized poly(ethylene glycol), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), acrylate-functionalized gelatin, methacrylate-functionalized gelatin, acrylate-functionalized hyaluronic acid, and methacrylate-functionalized hyaluronic acid; and
wherein at least one non-degradable crosslinker is functionalized with at least one functional moiety selected from the group consisting of acrylate, methacrylate, norbornene, thiol, azide, alkene, alkyne, oxime, hydrozone, isocyanate tetrazine, maleimide, vinyl sulphone, dibenzocyclooctyne, and NHS-ester.

79. The method of claim 73, wherein the at least one degradable crosslinker is an enzyme-degradable crosslinker, a protease-degradable crosslinker, a photodegradable crosslinker, and/or a biodegradable crosslinker.

80. The method of claim 73, wherein the at least one degradable crosslinker comprises at least one selected from the group consisting of ortho-nitrobenzyl moieties, coumarin, azobenzene, rotaxane, aromatic disulfides, poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), spermine, 2,2′-(ethylenedioxy)bis(ethylamine) (EDBE), CGPQGIWGQGC peptide, GPQGIAGQ peptide (PCL-1) and IPVSLRSG peptide (PCL-2).

81. The method of claim 73, wherein the at least one degradable crosslinker is a compound of Formula (II): 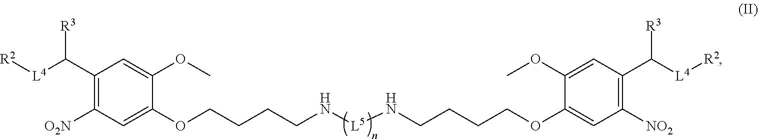
wherein:
each instance of L⁴ independently comprises a linkage having a structure selected from the group consisting of: 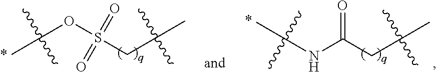
wherein the * side of the linkage is bound to the monomer and the opposite side is bound to R², and wherein q is an integer selected from 0 to 6;
L⁵ is a polymeric linker moiety comprising at least one selected from the group consisting of polyethylene glycol (PEG), poly(ethylene oxide), poly(vinyl alcohol), poly(vinyl acetate), poly(ethylene imine), polyacrylamide, poly(hyroxylethyl methacrylate), poly(N-vinyl pyrrolidone), poly(methacrylic acid), poly(butyl methacrylate), poly(methyl methacrylate), poly(meth acrylic acid), poly(N-isopropyl acrylamide), poly(hydroxylethylmethacrylate), poly(glycerol sebacate) (PGS), polylactic-glycolic acid (PLGA), poly-lactic acid (PLA), poly-caprolactone (PCL), copolymers of polylactic-glycolic acid and poly-caprolactone (PCL-PLGA copolymer), copolymers of polyethylene glycol and poly-caprolactone (PEG-PCL copolymer), copolymers of polyethylene glycol and trimethylene carbonate (PEG-TMC copolymer), copolymers of polyethylene glycol and poly(glycerol sebacate) (PEG-PGS copolymer), copolymers of polylactic-glycolic acid and poly-lactic acid (PLGA-PLA copolymer), polyhydroxy-butyrate-valerate (PHBV), polyorthoester (POE), polyethylene oxide-butylene terephthalate (PEO-PBTP), and poly-D,L-lactic acid-p-dioxanone-polyethylene glycol block copolymer (PLA-DX-PEG);
each instance of R² independently comprises a functionality selected from the group consisting of acrylate, methacrylate, alpha-methacrylate, norbornene, thiol, tetrazine, amine, dibenzocyclooctyne, maleimide, succinimide, trans-cyclooctene, azide, alkene, alkyne, oxime, hydrazone, alcohol, isocyanate, 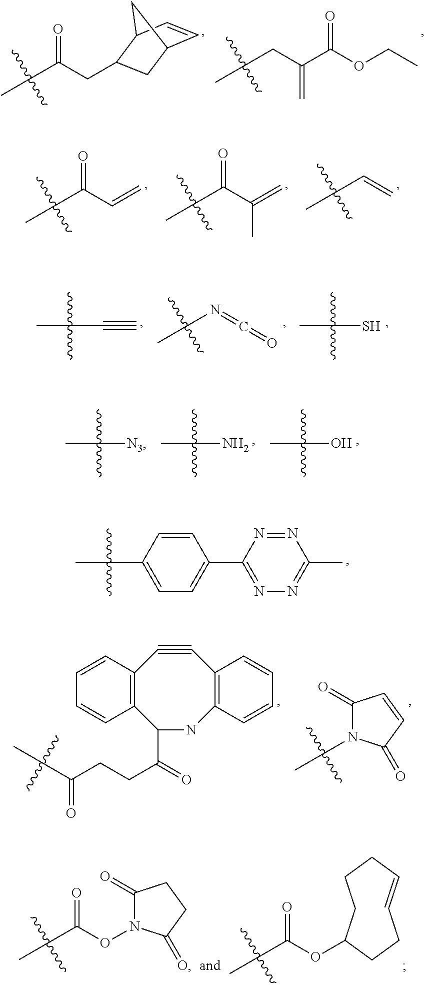
R³ is selected from the group consisting of H and methyl; and
n is an integer from 1 to 500.

82. The method of claim 73, wherein the polymer microspheres further comprise at least one magnetic particle having a diameter of about 100 nm to about 500 nm.

83. The method of claim 82, wherein the aggregation of portions of the uniformly disperse polymer microsphere composition comprises magnetically levitating the microspheres to form aggregates.

84. The method of claim 73, wherein the polymer microspheres are solid microspheres and/or core-shell particles comprising an outer shell and a hollow interior; and
wherein the cells are cultured on the inner surface of the outer shell, the cells are embedded within the polymer microspheres, and/or the cells are cultured on the surface of the polymer microspheres.

85. The method of claim 73, wherein at least one applies; (a) the polymer microspheres are monodisperse microspheres; (b) the polymer microspheres are fabricated through the use of a microfluidics device; (c) the polymer microspheres have a diameter of about 10 μm to about 300 μm; (d) the polymer microspheres have a diameter of about 200 μm; (e) the polymer microspheres have a stiffness of about 1 kPa to about 100 kPa; (f) the polymer microspheres have a stiffness of about 1 kPa to about 5 kPa; (g) the polymer microspheres have a stiffness of about 20 kPa to about 100 kPa; (h) the encapsulating matrix material has a stiffness of about 1 kPa to about 100 kPa; (i) the encapsulating matrix material has a stiffness of about 1 kPa to about 5 kPa or about 20 kPa to about 100 kPa; (j) the stiffness of the encapsulating matrix material is further adjusted using a dual stage curing process, (k) the polymer microspheres are fabricated through emulsion polymerization.

86. The method of claim 73, wherein incubating the cells in the encapsulating matrix degrades the degradable crosslinkers, thereby degrading the polymer microspheres while leaving the encapsulating matrix intact; or
wherein the at least one degradable crosslinker of the polymer microspheres is degraded through exposure to at least one selected from visible light (380 nm-760 nm) photoexcitation and ultraviolet (UV) light photoexcitation (100 nm-380 nm) thereby degrading the polymer microspheres while leaving the encapsulating matrix intact.

87. The method of claim 73, wherein the method further comprises testing the encapsulated cells for the presence of one or more biological markers.

88. The method of claim 73, further comprising adjusting the elastic modulus of the encapsulating matrix material using a dual stage curing process, wherein the dual stage curing process comprises a first polymerization stage and a second polymerization stage, the encapsulating matrix material has a greater elastic modulus after the second polymerization stage compared to prior to the second polymerization stage; and
wherein the encapsulating matrix material comprises an off-stoichiometric amount of the at least one multifunctional monomer and the at least one crosslinker where the amount of the at least one multifunctional monomer is greater than the at least one crosslinker.

89. The method of claim 88, wherein the at least one multifunctional monomer comprises the functional group 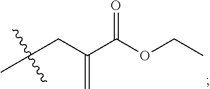 [SMILES: *CC(=C)C(=O)OCC]
and
the at least one crosslinker comprises the functional group

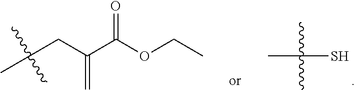

90. The method of claim 88, wherein the at least one crosslinker of the encapsulating matrix material comprises at least one non-degradable crosslinker and at least one degradable crosslinker, wherein the method further comprises reducing the elastic modulus of the encapsulating matrix material after the dual stage curing process by degrading the at least one degradable crosslinker.

91. An encapsulating matrix material composition, comprising;
at least one multifunctional monomer, and optionally further comprising at least one crosslinker, or at least one crosslinker and at least one peptide segment, and when present, the at least one crosslinker is in an off-stoichiometric amount in relation to the amount of the at least one multifunctional monomer;
wherein the at least one multifunctional monomer is a compound of Formula (IA): 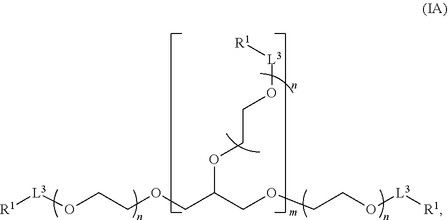
wherein:
each instance of L³ independently comprises a linkage selected from the group consisting of a bond, 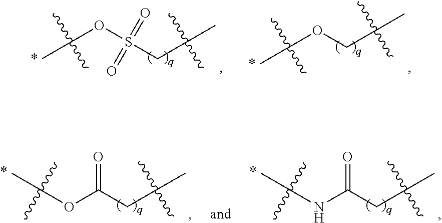
wherein the * side of the linkage is bound to the monomer and the opposite side is bound to R¹, and wherein q is an integer selected from 0 to 6;
each instance of R¹ independently comprises a functionality selected from the group consisting of acrylate, methacrylate, alpha-methacrylate, norbornene, thiol, azide, alkene, alkyne, oxime, hydrozone, isocyanate tetrazine, maleimide, vinyl sulphone, dibenzocyclooctyne, NHS-ester. 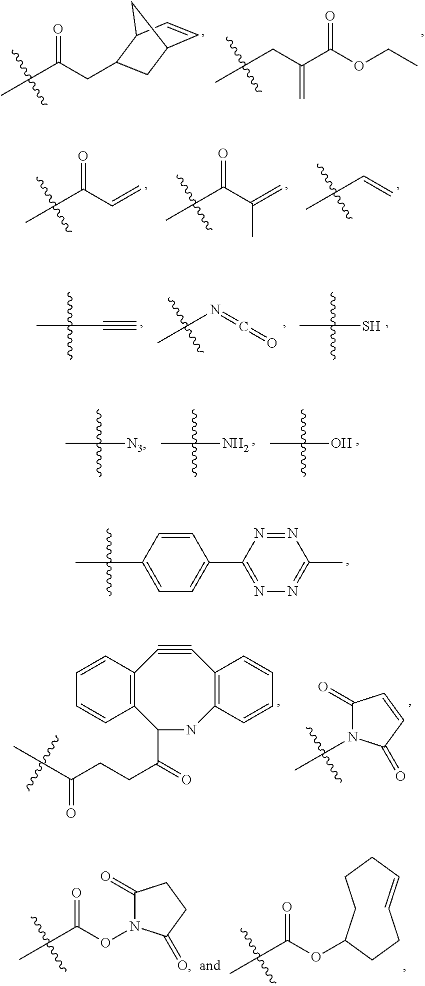
wherein at least one instance of R¹ is 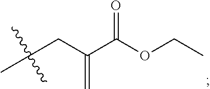 [SMILES: *CC(=C)C(=O)OCC]
m is an integer from 0 to 10; and
n is an integer from 1 to 500.

92. An aggregated alveoli-like structure, comprising alveoli-like clusters comprising at least one polymer microsphere composition, wherein the at least one polymer microsphere composition comprises at least one multifunctional monomer, at least one peptide segment, at least one degradable crosslinker, and optionally further comprising at least one cell; and
the alveoli-like clusters are encapsulated by the encapsulating matrix material composition of claim 93.
